(19) United States
(12) Patent Application Publication
(10) Pub. No.: US 2011/0287468 A1
(43) Pub. Date: Nov. 24, 2011

(54) METHODS AND COMPOSITIONS FOR ASSAYING ENZYMATIC ACTIVITY OF MYELOPEROXIDASE IN BLOOD SAMPLES

(75) Inventors: Chong-Sheng YUAN, San Diego, CA (US); Xiaomin GONG, San Diego, CA (US)

(73) Assignee: General Atomics, San Diego, CA (US)

(21) Appl. No.: 13/090,051

(22) Filed: Apr. 19, 2011

Related U.S. Application Data
(60) Provisional application No. 61/325,788, filed on Apr. 19, 2010.

(51) Int. Cl. C12Q 1/28

(52) U.S. Cl. 435/28

(57) ABSTRACT

The present invention provides a two-step assay for measuring myeloperoxidase (MPO) activity in a blood sample. The first step utilizes a chromogenic substrate to measure first peroxidase activity including MPO activity in the sample, whereas the second step measures non-MPO peroxidase activity in the presence of the same chromogenic substrate and a specific MPO inhibitor. Specific MPO peroxidase activity is then determined by comparing the non-MPO peroxidase activity and the total peroxidase activity. The MPO peroxidase activity obtained in this fashion may be proportional, and preferably directly proportional, to the mass of MPO in the sample. Kits for assaying MPO peroxidase activity based on the same principle are also provided.

### RELATED APPLICATION

This application claims benefit of priority to U.S. Provisional Application Ser. No. 61/325,788, filed Apr. 19, 2010, the content of which is incorporated herein by reference in its entirety.

### FIELD OF THE INVENTION

This invention generally relates to the field of myeloperoxidase (MPO) detection. In particular, the invention provides novel methods and kits for measuring the amount of MPO in blood samples.

### BACKGROUND OF THE INVENTION

Myeloperoxidase (MPO; EC 1.11.1.7) is a tetrameric, heavily glycosylated basic heme protein of approximately 150 kDa. It is composed of two identical disulfide-linked protomers, each of which possesses a protoporphyrin-containing 59-64 kDa heavy subunit and a 14 kDa light subunit. U.S. Pat. No. 7,223,552; Hoy et al., Clin. Chem. Lab. Med. 40: 2-8 (2002). In vivo, MPO converts chloride ions (Cl⁻) via a two-electron peroxidation step into hypochlorous acid, HOCl, a powerful oxidizing agent capable of destroying microbes. Marquez et al., J. Biol. Chem. 265: 5666-5670 (1990).

MPO plays an important role in host defense against invading microorganisms. MPO is abundant in neutrophils and monocytes, accounting for 5% and 1-2% of the dry weight of these cells, respectively. Marquez et al., J. Biol. Chem. 265: 5666-5670 (1990); U.S. Pub. No. 2002/0164662.

MPO is implicated in a broad spectrum of diseases. Besides participating in the defense against microorganisms via the production of HOCl, MPO is released in inflammatory states where migrating neutrophils may release active enzyme. Hoy et al., Clin. Chem. Lab. Med. 40: 2-8 (2002). Increased MPO levels have been reported in infections, and anti-MPO antibodies accumulate in systemic vasculitites. MPO is also involved in non-infectious diseases, such as atherosclerosis, cancer and promyelocytic leukemia, neurodegerative diseases including Alzheimer's disease and multiple sclerosis. Hoy et al., Clin. Chem. Lab. Med. 40: 2-8 (2002).

MPO mRNA is widely used in clinical chemistry as a marker for acute myeloid leukemia (AML). Bennett et al., Br. J. Haematol. 33: 451-8 (1976). Higher expression genotype of the MPO G-463A polymorphism has also been reported to be related to AML. Reynolds et al., Blood 90: 2730-7 (1997). The MPO G-463A polymorphism characterized by a G/A transition is located with Alu sequences of a promoter region containing a hormone response element. The G/G genotype has been related to increased MPO expression and protein level in cells of leukemic patients. Reynolds et al., Blood 90: 2730-7 (1997). It has also been reported that subjects homozygous for the A allele are at a decreased risk for lung cancer. London et al., Cancer Res. 57: 5001-3 (1997); Le Marchand et al., Cancer Epidermiol. Biomarkers Prev. 9: 181-4 (2000); Cascorb et al., Cancer Res. 60: 644-9 (2000); Schabath et al., Carcinogenesis 21: 1163-6 (2000). However, another study showed that the A allele is associated with an increased risk of lung cancer among a subset of older men. Misra et al., Cancer Lett. 164: 161-7 (2001).

MPO is present in the microglia in the brain of patients with multiple sclerosis (MS) and in the microglial cells surrounding senile plaques of cerebral cortex from Alzheimer's disease (AD) cases. Jolivalt et al., Neurosci. Lett. 210: 61-4 (1996); Nagra et al., J. Neuroimmunol. 78: 97-107 (1997). An alternation of MPO level has also been linked to atherosclerosis and stroke. Nicholls & Hazen, J. Lipid Res., 50: S346-351 (2009); U.S. Pat. No. 7,608,406. It has been reported that MPO/H₂O₂/Cl⁻ system is one of the possible mechanisms involved in the initiation of atherosclerotic lesions. Dautherty et al., J. Clin. Invest. 94: 437-44 (1994). Heinecke et al., Curr. Opinion Lip. 8: 268-74 (1997); Hazell et al., J. Clin. Invest. 97: 1535-44 (1996); Malle et al., Eur. J. Biochem. 267: 4495-503 (2000). One of the main consequences of atherosclerosis is brain infarction and measurement of MPO activity is a widely used marker of neutrophil infiltration of the brain parenchyma. Barone et al., J. Neuroscie. Res. 29: 336-45 (1991). Increased MPO activity has been observed in the serum of patients after an ischemic brain infarction. Azzimondi et al., Eur. J. Emerg. Med. 4: 5-9 (1997).

Because MPO is implicated in the pathogenesis of atherosclerosis, measurement of MPO has been used to predict various cardiovascular risks. Nicholls & Hazen, Arterioscler. Thromb. Vasc. Biol. 25: 1102-1111 (2005). For example, MPO levels in blood have been used as diagnostic and predictive markers for coronary arterial disease (Baldus et al., Circulation, 108: 1440-1445 (2003); Brennan et al., N. Engl. J. Med. 349: 1595-1601 (2003); U.S. Pat. No. 7,223,552), peripheral arterial disease (Ali et al., Vasc. Med., 14: 215-220 (2009)), heart failure (Tang et al., Am. J. Cardiol. 103: 1269-1274 (2009)) and acute myocardial infarction (Chang et al., Circ. J., 73: 726-731 (2009)).

The broad range of pathologic conditions in which MPO is implicated and the possibility of using MPO as a clinical marker and therapeutic target make assays for accurately measuring MPO levels and activities invaluable. A number of different MPO assays have been disclosed in U.S. Pat. Nos. 6,022,699; 7,108,997; 7,195,891; U.S. Pat. Pub. No. 2009/0162876, all of which are incorporated herein by reference. Most MPO assays are based on either immunodetection or measurement of enzymatic activity.

MPO immunoassays are available from multiple commercial sources (e.g., Calbiochem® Myeloperoxidase ELISA Kit, EMD Chemicals, Inc.; PLAC® Test, diaDexus, Inc.; CardioMPO® Test, PrognostiX, Inc.). The PrognostiX CardioMPO® assay has been licensed to Abbott Laboratories, Inverness Medical Innovations and Siemens Medical Solutions, some of which incorporated it into proprietary automated immunodiagnostic systems (e.g., Siemens Dimension® RxL Max® and XPand Plus®, see Shah et al., Clin. Chem. 55: 59-67 (2008); and Abbott Diagnostics Architect® MPO Assay, see Zelzer et al., Clin. Chim. Acta, 406: 62-65 (2009)). However, most clinical laboratories cannot afford these proprietary automated systems and must rely on conventional clinical chemistry analyzers instead. Unfortunately, there are currently no MPO ELISA assays that are compatible with such analyzers.

Enzymatic MPO assays have been known for over forty years. See, e.g., Klebanoff, J. Bacteriol., 95: 2132-2138 (1968). These assays usually involve the use of a chromogenic substrate in combination with hydrogen peroxide, wherein the substrate is oxidized by MPO to produce an optically detectable product. Commonly used substrates include o-dianisidine (DA) (Rosen & Klebanoff, J. Clin. Invest., 58: 50-60 (1976)), 3,3′,5,5′-tetramethylbenzidine (TMB) (Suzuki et al., Anal. Biochem., 132: 345-352 (1983)), and other aromatic molecules, such as guaiacol, 4-chloro-naphthol and tyrosine (Gorudko et al., Rus. J. Bioorg. Chem., 35: 566-575 (2009)). However, the measurement of MPO peroxidase activity in blood samples is complicated by the fact that plasma contains numerous oxidizing and reducing components other than MPO (e.g., non-MPO peroxidases, hemoglobin, glutathione, ascorbate etc.) that act on the same substrate and/or otherwise interfere with MPO peroxidase activity.

Previous attempts to reduce or eliminate non-MPO contributions to peroxidase activity have been reported in the art. For example, gel filtration was used to measure MPO activity in a tissue sample. Xia & Zweier, Anal. Biochem. 245: 93-96 (1997). Obviously, gel filtration is a slow and labor-intensive technique that is impractical to use in routine clinical testing. In a different study, a specific MPO inhibitor, 4-aminobenzoic acid hydrazide (ABAH), was used for selective determination of MPO activity in equine synovial fluid, wherein non-MPO peroxidase activity was subtracted from total peroxidase activity to obtain MPO peroxidase activity. Fietz et al., Res. Vet. Sci., 84: 347-353 (2008). The same approach and MPO inhibitor were used in another study to measure MPO peroxidase activity in human blood plasma. Gorudko et al., Rus. J. Bioorg. Chem., 35: 566-575 (2009). However, this assay only worked under acidic conditions (pH 4-5) and exhibited very low peroxidase activity at pH 5.5 or greater. Since it had previously been shown that MPO exhibits dominant peroxidase activity at neutral pH and dominant chlorinating activity at acidic pH (Vlasova et al., Biochemistry (Moscow), 71: 667-677 (2006)), it appears that a peroxidase assay that only works at acidic pH may not be optimal.

Accordingly, there remains a need for a reliable, sensitive and specific method for measuring MPO peroxidase activity in blood samples, particularly one that can be performed at approximately neutral pH and is amenable to automation in the typical clinical laboratory settings.

### BRIEF SUMMARY OF THE INVENTION

In one aspect, the present invention provides methods for measuring a myeloperoxidase (MPO) activity in a blood sample, the methods comprising: a) contacting a blood sample containing or suspected of containing MPO with a chromogenic MPO substrate that minimizes interferences of the MPO activity in the blood sample, and a non-chromogenic co-substrate for MPO to measure a first peroxidase activity in the blood sample, wherein the chromogenic MPO substrate is not o-dianisidine; b) contacting the blood sample with the chromogenic MPO substrate, the non-chromogenic co-substrate for MPO and a specific MPO activity inhibitor to measure a second peroxidase activity in the blood sample; and c) comparing the first and second peroxidase activities to obtain MPO activity in the blood sample. In some embodiments, the blood sample is selected from whole blood, serum and plasma from which substantially all hemoglobin has been removed, preferably from human whole blood, serum or plasma from which substantially all hemoglobin has been removed. In some embodiments, the assay specifically measures secreted MPO activity in human serum or plasma.

Any suitable chromogenic MPO substrates that minimize interferences of the MPO activity in a blood sample can be used in the present methods. In some embodiments, the chromogenic MPO substrate minimizes interferences of the MPO activity in a human blood sample, such as human serum or plasma from which substantially all hemoglobin has been removed. In some embodiments, the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline. Preferably, the salts of N-ethyl-N-(2-hydroxy-3-sulfopropyl)-3-methylaniline, N,N-bis(4-sulfobutyl)-3-methylaniline, 3,5-dichloro-2-hydroxybenzenesulfonate, N-ethyl-N-(3-sulfopropyl)-3-methylaniline and N-ethyl-N-(3-sulfopropyl)-aniline are sodium or disodium salts.

Any suitable non-chromogenic co-substrate for MPO can be used in the present methods. In some embodiments, the non-chromogenic co-substrate for MPO comprises hydrogen peroxide (H₂O₂) and/or 4-aminoantipyrine (4-AA). Any suitable specific MPO activity inhibitors can be used in the present methods. In some embodiments, the specific MPO activity inhibitor is a benzoic acid hydrazide such as 4-aminobenzoic acid hydrazide (ABAH) (Kettle et al., Biochem., 308: 559-563 (1995)), a hydroxamic acid such as benzohydroxamic acid (BHA), a salicylhydroxamic acid (SHA) (Davies & Edwards, Biochem. J., 258: 801-806 (1989)), a thioxanthine derivative such as 3-n-propyl-2-thioxanthine, 3-isobutyl-6-thioxanthine and other thioxanthine derivatives disclosed in U.S. Pat. No. 7,425,560, U.S. Pat. Appl. Nos. 2007/0032468 and 2009/0124640, Int'l Pub. Nos. WO 01/85146, WO 03/089430 and WO 05/037835, Jacobson et al., Drug. Dev. Res., 47: 45-53 (1999) and Wooldridge & Slack, J. Chem. Soc., 1863 (1962), or a 2,4-dihydro-[1,2,4]triazole-3-thione derivative disclosed in U.S. Pat. Appl. No. 2007/0093483, all of which are incorporated herein by reference. In preferred embodiments, the specific MPO activity inhibitor is 4-aminobenzoic acid hydrazide (ABAH).

The first peroxidase activity and/or the second peroxidase activity can be measured by any suitable methods or means. In some embodiments, the first peroxidase activity and/or the second peroxidase activity are measured by measuring the oxidative product of the chromogenic MPO substrate. In some embodiments, the oxidative product of the chromogenic MPO substrate is detectable in the visible region of the electromagnetic spectrum (380-760 nm) and preferably measured by a spectrometer or a spectrophotometer. In other embodiments, the first peroxidase activity and/or the second peroxidase activity are measured by measuring the reduction of the chromogenic MPO substrate and/or the non-chromogenic co-substrate for MPO.

The first and second peroxidase activities can be compared in any suitable way to obtain the MPO activity in the blood sample, e.g., comparing any suitable additive, subtractive, multiplying, dividing, ratio or proportion values of the first and second peroxidase activities. Preferably, the step of comparing the first and second peroxidase activities comprises subtracting the second peroxidase activity from the first peroxidase activity to obtain the MPO activity in the blood sample.

Any suitable blood sample can be assayed by the present methods. Preferably, the blood sample is pre-treated before the assay by removing substantially all hemoglobin (i.e., red blood cells) in order to eliminate or significantly reduce the oxidative interference from the hemoglobin molecules. In some embodiments, the blood sample is selected from human serum or plasma; the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH). Preferably, the chromogenic MPO substrate is selected from 3,5-dichloro-2-hydroxybenzenesulfonate, a salt of 3,5-dichloro-2-hydroxybenzenesulfonate and 3,5-dichloro-2-hydroxybenzenesulfonic acid.

As noted above, the present methods are preferably carried out at approximately neutral pH. Thus, the contacting steps a) and/or b) are preferably conducted at a pH that ranges from about 5.0 to about 8.0, more preferably from about 5.5 to about 7.5, and most preferably from about 6.0 to about 7.0.

The present methods can be conducted in any suitable format. In some embodiments, the methods of the present invention are conducted in a homogenous assay format. Alternatively, the methods of the present invention may be conducted in a heterogeneous assay format. Preferably, the assay is automated; however manual operation is also possible and contemplated within the present invention.

The present methods can be used for any suitable purpose. In some embodiments, the present methods may be used for prognosis, diagnosis and/or monitoring treatment of a disease, such as coronary arterial disease, peripheral arterial disease, heart failure, acute myocardial infarction, atherosclerosis, stroke, multiple sclerosis, Alzheimer's disease, lung cancer, leukemia, or microbial infection.

In another aspect, the present invention provides kits for measuring a myeloperoxidase (MPO) activity in a blood sample, the kits comprising a chromogenic MPO substrate that minimizes interferences of the MPO activity in a blood sample, wherein said chromogenic MPO substrate is not o-dianisidine, a non-chromogenic co-substrate for MPO, and a specific MPO activity inhibitor.

Any suitable chromogenic MPO substrates that minimize interferences of the MPO activity in a blood sample can be used in the present kits. In some embodiments, the chromogenic MPO substrate minimizes interferences of the MPO activity in a human blood sample, such as whole blood, serum or plasma from which substantially all hemoglobin has been removed, preferably serum or plasma. In some embodiments, the assay specifically measures secreted MPO activity in human serum or plasma.

In some embodiments, the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline. Preferably, the salts of N-ethyl-N-(2-hydroxy-3-sulfopropyl)-3-methylaniline, N,N-bis(4-sulfobutyl)-3-methylaniline, 3,5-dichloro-2-hydroxybenzenesulfonate, N-ethyl-N-(3-sulfopropyl)-3-methylaniline and N-ethyl-N-(3-sulfopropyl)-aniline are sodium or disodium salts.

Any suitable non-chromogenic co-substrate for MPO can be used in the present kits. In some embodiments, the non-chromogenic co-substrate for MPO comprises hydrogen peroxide (H₂O₂) and/or 4-aminoantipyrine (4-AA). Any suitable specific MPO activity inhibitors can be used in the present kits. In some embodiments, the specific MPO activity inhibitor is a benzoic acid hydrazide such as 4-aminobenzoic acid hydrazide (ABAH) (Kettle et al., Biochem., 308: 559-563 (1995)), a hydroxamic acid such as benzohydroxamic acid (BHA), a salicylhydroxamic acid (SHA) (Davies & Edwards, Biochem. J., 258: 801-806 (1989)), a thioxanthine derivative such as 3-n-propyl-2-thioxanthine, 3-isobutyl-6-thioxanthine and other thioxanthine derivatives disclosed in U.S. Pat. No. 7,425,560, U.S. Pat. Appl. Nos. 2007/0032468 and 2009/0124640, Int'l Pub. Nos. WO 01/85146, WO 03/089430 and WO 05/037835, Jacobson et al., Drug. Dev. Res., 47: 45-53 (1999) and Wooldridge & Slack, J. Chem. Soc., 1863 (1962), or a 2,4-dihydro-[1,2,4]triazole-3-thione derivative disclosed in U.S. Pat. Appl. No. 2007/0093483, all of which are incorporated herein by reference. In preferred embodiments, the specific MPO activity inhibitor is 4-aminobenzoic acid hydrazide (ABAH).

Any suitable blood sample can be assayed by the present kits. Preferably, the blood sample is pre-treated before the assay by removing substantially all hemoglobin (i.e. red blood cells) in order to eliminate or significantly reduce the oxidative interference from the hemoglobin molecules. In some embodiments, the blood sample is selected from human serum or plasma; the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH). Preferably, the chromogenic MPO substrate is selected from 3,5-dichloro-2-hydroxybenzenesulfonate, a salt of 3,5-dichloro-2-hydroxybenzenesulfonate and 3,5-dichloro-2-hydroxybenzenesulfonic acid.

In some embodiments, the present kits further comprise a means for measuring the oxidative product of the chromogenic MPO substrate, such as a spectrometer or a spectrophotometer capable of measuring optical signals having wavelengths in the visible region of the electromagnetic spectrum (380-760 nm).

The present kits can be used for any suitable purpose. In some embodiments, the present kits may be used for prognosis, diagnosis and/or monitoring treatment of a disease, such as coronary arterial disease, peripheral arterial disease, heart failure, acute myocardial infarction, atherosclerosis, stroke, multiple sclerosis, Alzheimer's disease, lung cancer, leukemia, or microbial infection.

In some embodiments, the present invention provides for a method for measuring a myeloperoxidase (MPO) activity in a blood sample, which method comprises: a) contacting a blood sample containing or suspected of containing MPO with a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline, and a non-chromogenic co-substrate for MPO to measure a first peroxidase activity in said blood sample; b) contacting said blood sample with said chromogenic MPO substrate, said non-chromogenic co-substrate for MPO and a specific MPO activity inhibitor to measure a second peroxidase activity in said blood sample; and c) comparing said first and second peroxidase activities to obtain the MPO activity in said blood sample.

In other embodiments, the present invention provides for a kit for measuring a myeloperoxidase (MPO) activity in a blood sample, which kit comprises: a) a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; b) a non-chromogenic co-substrate for MPO; and c) a specific MPO activity inhibitor.

The present methods can be conducted in any suitable format, e.g., single channel, dual channel or multiple channel assay format. In some embodiments, the present methods can be conducted in a single channel assay format. For example, the first peroxidase activity and the second peroxidase activity can be measured in a single channel sequentially, the first peroxidase activity being measured in the presence of the chromogenic MPO substrate that minimizes interferences of the MPO activity in the blood sample and the non-chromogenic co-substrate for MPO to measure a first total peroxidase activity in the blood sample, the second peroxidase activity being measured by adding the specific MPO activity inhibitor after the first peroxidase activity is measured to measure a second non-MPO peroxidase activity in the blood sample, and the MPO activity in the blood sample is obtained by subtracting the second non-MPO peroxidase activity from the first total peroxidase activity. In a specific embodiment, the first peroxidase activity is measured after addition of reagent 1 comprising the chromogenic MPO substrate and reagent 2 comprising the non-chromogenic co-substrate for MPO to the blood sample, and the second peroxidase activity is measured after addition of reagent 3 comprising the specific MPO activity inhibitor to the blood sample after the first peroxidase activity is measured.

In other embodiments, the present methods can be conducted in dual channel assay format. For example, the first peroxidase activity and the second peroxidase activity can be measured in two channels separately, the first peroxidase activity being measured in the presence of the chromogenic MPO substrate that minimizes interferences of the MPO activity in the blood sample and the non-chromogenic co-substrate for MPO in a first channel to measure a first total peroxidase activity in the blood sample, the second peroxidase activity being measured in the presence of the chromogenic MPO substrate that minimizes interferences of the MPO activity in the blood sample, the non-chromogenic co-substrate for MPO and the specific MPO activity inhibitor in a second channel to measure a second non-MPO peroxidase activity in the blood sample, and the MPO activity in the blood sample is obtained by subtracting the second non-MPO peroxidase activity from the first total peroxidase activity. In a specific embodiment, the first peroxidase activity is measured after addition of the reagent comprising the chromogenic MPO substrate and the reagent comprising the non-chromogenic co-substrate for MPO to the blood sample, and the second peroxidase activity is measured after addition of the reagent comprising the chromogenic MPO substrate and the specific MPO activity inhibitor and the reagent comprising the non-chromogenic co-substrate for MPO to the blood sample.

The present kits can be formulated to be used in any suitable format, e.g., single channel, dual channel or multiple channel assay format. In some embodiments, the present kits can be formulated to be used in a single channel assay format. For example, the kit can comprise the following reagents: a) reagent 1 comprising the chromogenic MPO substrate; b) reagent 2 comprising the non-chromogenic co-substrate for MPO; and c) reagent 3 comprising the specific MPO activity inhibitor. In other embodiments, the present kits can be formulated to be used in a dual channel assay format. For example, the kit can comprise the following reagents: a) reagent 1 comprising the chromogenic MPO substrate; b) reagent 2 comprising the chromogenic MPO substrate and the specific MPO activity inhibitor; and c) reagent 3 comprising the non-chromogenic co-substrate for MPO.

The reagents can comprise other substances for various purposes. The exemplary substances can include, but are not limited to cyclodrextrin and derivatives, Dextran, D-sorbital, BSA, EGTA, EDTA, K4Fe(CN)6, sodium cholate, sodium citrate, Triton X-100, 4-hydroxy-TEMPO, sodium benzoate, ascorbate oxidase, and Tris-HCl.

### BRIEF DESCRIPTION OF THE DRAWINGS

FIG. 1 illustrates the effect of a specific MPO inhibitor, 4-aminobenzoic acid hydrazide (ABAH), on MPO peroxidase activity measured using four chromogenic MPO substrates of the present invention, TOOS/4AA, TODB/4AA, ALPS/4AA and DHBS/4AA.

FIG. 2 illustrates the correlation between single channel and duel channel assay formats. The performance of the dual channel MPO assay was compared with the performance of the single channel MPO assay using lithium heparin plasma samples ranging from 21 to 1300 ng/mL (146-9022 pmol/L). For the total of 38 samples tested, the correlation coefficient between the two methods is 0.9788; the slope is 0.9697; and y intercept is 11.169 ng/mL.

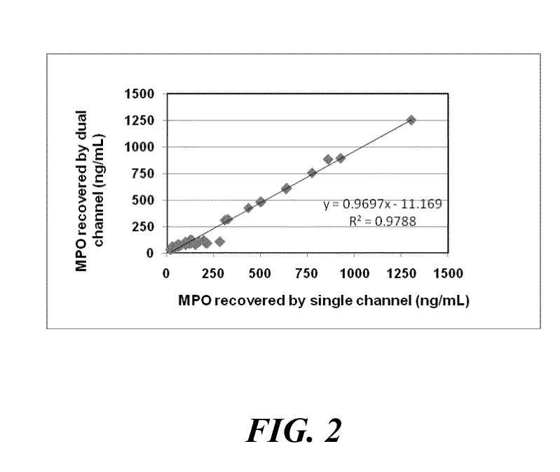

### DETAILED DESCRIPTION OF THE INVENTION

The present invention provides a two-step assay for measuring myeloperoxidase (MPO) activity. The first step utilizes a chromogenic substrate to measure first peroxidase activity in a sample, whereas the second step measures a second, non-MPO peroxidase activity in the presence of the same chromogenic substrate and a specific MPO inhibitor. Specific MPO peroxidase activity is then determined by comparing the first and second peroxidase activities, e.g., subtracting the non-MPO peroxidase activity from the first peroxidase activity. In some embodiments, the MPO peroxidase activity obtained in this fashion is proportional, preferably directly proportional, to the mass of MPO in the sample.

For clarity of disclosure, and not by way of limitation, the detailed description of the invention is divided into the subsections that follow.

### A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “myeloperoxidase” refers to an enzyme, classified as EC 1.11.1.7 according to International Union of Biochemistry and Molecular Biology (IUBMB) enzyme classification, which catalyzes formation of an oxidized donor and H₂O from the donor and H₂O₂. For example, myeloperoxidase catalyzes formation of HOCl and H₂O from Cl⁻ and H₂O₂. It is intended to encompass derivatives, variants, and analogs of myeloperoxidase that do not substantially alter its activity. Myeloperoxidase can be obtained from any source, such as human, mouse, bovine, rat, fruit fly, etc.

As used herein, the term “measuring” is intended to include both quantitative and qualitative determination in the sense of obtaining an absolute value for the amount or concentration of the analyte present in the reaction system, and also of obtaining an index, ratio, percentage, visual or other value indicative of the level of analyte in the reaction system. Measurement may be direct or indirect, and the chemical species actually detected need not be the analyte itself but may be a derivative thereof or some other substance.

As used herein, “blood sample” refers to refers to a whole blood sample or a plasma or serum fraction derived therefrom. Preferably, the blood sample refers to a human blood sample such as whole blood or a plasma or serum fraction derived therefrom. Also preferably, the blood sample is pre-treated before the assay by removing substantially all hemoglobin (i.e. red blood cells) in order to eliminate or significantly reduce the oxidative interference from the hemoglobin molecules.

As used herein the term “whole blood” refers to a blood sample that has not been fractionated and contains both cellular and fluid components. As used herein, “whole blood” refers to freshly drawn blood which is tested before it clots, or a conventionally-drawn blood sample, which may be drawn into a vacutainer, and which may contain an anticoagulant, such as lithium-heparin, EDTA etc., or to which one or more other standard clinical agents may be added in the course of routine clinical testing.

As used herein, the phrase “substantially all hemoglobin has been removed” refers to a blood sample wherein preferably at least about 50%, 60% or 70%, more preferably, at least about 80%, 90% or 95%, and most preferably, at least about 96%, 97%, 98%, 99 or 100% of all hemoglobin-containing red blood cells in the sample have been removed to eliminate or significantly reduce the oxidative interference from hemoglobin.

As used herein, the term “plasma” refers to the fluid, non-cellular component of the whole blood. Depending on the separation method used, plasma may be completely free of cellular components, or may contain various amounts of platelets and/or a small amount of other cellular components. Because plasma includes various clotting factors such as fibrinogen, the term “plasma” is distinguished from “serum” as set forth below.

As used herein, the term “serum” refers to whole mammalian serum, such as whole human serum. Further, as used herein, “serum” refers to blood plasma from which clotting factors (e.g., fibrinogen) have been removed.

As used herein, the term “fluid” refers to any composition that can flow. Fluids thus encompass compositions that are in the form of semi-solids, pastes, solutions, aqueous mixtures, gels, lotions, creams and other such compositions.

As used herein, the term “disease” or “disorder” refers to a pathological condition in an organism resulting from, e.g., infection or genetic defect, and characterized by identifiable symptoms.

As used herein, “contacting” means bringing two or more components together. “Contacting” can be achieved by mixing all the components in a fluid or semi-fluid mixture. “Contacting” can also be achieved when one or more components are brought into contact with one or more other components on a solid surface such as a solid tissue section or a substrate.

As used herein, the term “chromogenic substrate” refers to a chemical composition that can participate in a particular enzymatic reaction as either a donor or an acceptor for the reaction and that changes color during the reaction. For example, myeloperoxidase converts hydrogen peroxide to water by borrowing two hydrogen atoms from a donor molecule. When the donor molecule is a chromogenic substrate, the oxidation of the chromogenic substrate causes the substrate to change to a detectable color. For example, 3,3′,5,5′-tetramethylbenzidine (TMB) is colorless in the reduced state but blue in the oxidized state or yellow in the diamine state.

As used herein, the term “non-chromogenic co-substrate” refers to a chemical composition that participates in the same enzymatic reaction as the chromogenic substrate but does not change color during the reaction. In the example above, hydrogen peroxide is a non-chromogenic co-substrate because both water and hydrogen peroxide are colorless.

As used herein, the term “specific MPO inhibitor” refers to chemical compositions that selectively inhibit MPO peroxidase activity without significantly affecting the enzymatic activities of non-MPO peroxidases in a blood sample. Preferably, the specific MPO inhibitor inhibits at least about 50%, 60% or 70%, more preferably, at least about 80%, 90% or 95%, and most preferably, at least about 96%, 97%, 98%, 99% or 100% of the specific MPO peroxidase activity in a blood sample. Also preferably, the specific MPO inhibitor inhibits less than about 50%, 40% or 30%, more preferably, less than about 20%, 10% or 5%, and most preferably, less than about 4%, 3%, 2% or 1% of the non-MPO peroxidase activity in a blood sample. As noted above, known examples of specific MPO inhibitors include benzoic acid hydrazides such as 4-aminobenzoic acid hydrazide (ABAH) (Kettle et al., Biochem., 308: 559-563 (1995)), hydroxamic acids such as benzohydroxamic acid (BHA) and salicylhydroxamic acid (SHA) (Davies & Edwards, Biochem. J., 258: 801-806 (1989)), and thioxanthine derivatives such as 3-n-propyl-2-thioxanthine, 3-isobutyl-6-thioxanthine and other thioxanthine derivatives disclosed in U.S. Pat. No. 7,425,560, U.S. Pat. Appl. Nos. 2007/0032468 and 2009/0124640, Int'l Pub. Nos. WO 01/85146, WO 03/089430 and WO 05/037835, Jacobson et al., Drug. Dev. Res., 47: 45-53 (1999) and Wooldridge & Slack, J. Chem. Soc., 1863 (1962), and 2,4-dihydro-[1,2,4]triazole-3-thione derivatives disclosed in U.S. Pat. Appl. No. 2007/0093483.

As used herein, the phrase “a chromogenic MPO substrate that minimizes interferences of the MPO activity in said blood sample” refers to a chromogenic MPO substrate that decreases the amount of optical, oxidative and/or reductive activity in the blood sample that is mediated by blood components other than MPO (e.g., glutathione, ascorbic acid, non-MPO peroxidases, etc.) and/or specific MPO inhibitors (e.g., ABAH), when compared to data obtained using chromogenic substrates of the prior art. Preferably, the chromogenic substrate reduces nonspecific optical, oxidative and/or reductive activity in the blood sample by about 5%, 10%, 20% or 30%, more preferably by about 40%, 50%, 60% or 70%, and most preferably by about 80%, 90%, 95% or 99% relative to chromogenic substrates of the prior art, such as for example, o-dianisidine (DA) or 3,3′,5,5′-tetramethylbenzidine (TMB).

As used herein, the term “comparing” generally means examining in order to note similarities or differences between two or more values. Preferably, “comparing” refers to quantitative comparisons such as, for example, subtracting one value from another, calculating a ratio of two values, calculating a percentage of one value with respect to another, or combining these types of calculations to produce a single number. As used herein, “comparing” further refers to comparisons made by a human, comparisons made by a computer or other processor, and comparisons made by a human in combination with a computer or other processor.

### B. Methods for Assaying Myeloperoxidase

As noted above, the present invention provides methods for measuring a myeloperoxidase (MPO) activity in a blood sample, the methods comprising: a) contacting a blood sample containing or suspected of containing MPO with a chromogenic MPO substrate that minimizes interferences of the MPO activity in the blood sample, and a non-chromogenic co-substrate for MPO to measure a first peroxidase activity in the blood sample, wherein the chromogenic MPO substrate is not o-dianisidine; b) contacting the blood sample with the chromogenic MPO substrate, the non-chromogenic co-substrate for MPO and a specific MPO activity inhibitor to measure a second peroxidase activity in the blood sample; and c) comparing the first and second peroxidase activities to obtain MPO activity in the blood sample. In some embodiments, the blood sample is selected from whole blood, serum and plasma from which substantially all hemoglobin has been removed, preferably from human whole blood, serum or plasma from which substantially all hemoglobin has been removed. In some embodiments, the assay specifically measures secreted MPO activity in human serum or plasma. In some embodiments, the step of comparing the first and second peroxidase activities comprises subtracting the second peroxidase activity from the first peroxidase activity to obtain the MPO activity in the blood sample.

Step a): Contacting a Blood Sample Containing or Suspected of Containing MPO with a Chromogenic MPO Substrate that Minimizes Interferences of the MPO Activity in the Blood Sample, and a Non-Chromogenic Co-Substrate for MPO to Measure a First Peroxidase Activity in the Blood Sample, Wherein the Chromogenic MPO Substrate is not o-Dianisidine

The present methods measure MPO peroxidase activity in a blood sample via a two-step process. In the first step, a first peroxidation activity is measured through the reaction of various peroxidases including MPO in the blood sample using a chromogenic substrate that minimizes interferences of the MPO activity in the blood sample, wherein the chromogenic MPO substrate is not o-dianisidine, and a non-chromogenic MPO substrate. Preferably, the chromogenic MPO substrate minimizes interferences of the MPO activity in a human blood sample, such as human whole blood, serum or plasma from which substantially all hemoglobin has been removed. The peroxidation reaction may be summarized as follows:

Peroxidases+2AH₂+H₂O₂→2*AH+2H₂O(total activity) (1)

wherein AH₂ denotes the peroxidase substrate, and *AH denotes the product of oxidation.

As discussed above, one of the main difficulties of measuring MPO peroxidase activity in blood samples is the presence of additional oxidizing or reducing substances that exhibit peroxidase activity or otherwise interfere with MPO-mediated peroxidation. Thus, one of the main objectives of the present invention was to identify those chromogenic substrates that minimize interferences of MPO peroxidase activity by blood components. To this end, a number of known or putative chromogenic substrates of MPO were analyzed. The names and optical properties of these substrates are summarized in Table 1.

TABLE 1
Chromogenic MPO substrates screened for MPO peroxidase activity

| | Substrate | | | |
|---|---|---|---|---|
| | Abbreviation | Wavelength | ε (m⁻¹cm⁻¹) | Chemical Name |
| 1 | Guaiacol | 470 nm | 2.6 × 10⁴ | 1-Hydroxy-2-methoxybenzene; 2- |
| | | | | Methoxyphenol |
| 2 | TMB | 655 nm | 3.9 × 10⁴ | 3,3′,5,5′-Tetramethylbenzidine |
| 3 | TOOS/4AA | 555 nm | 3.92 × 10⁴ | N-Ethyl-N-(2-hydroxy-3-sulfopropyl)-3- |
| | | | | methylaniline, sodium salt, dehydrate/4- |
| | | | | Aminoantipyrine (4AA) |
| 4 | TODB/4AA | 555 nm | 3.8 × 10⁴ | N,N-Bis(4-sulfobutyl)-3-methylaniline, |
| | | | | disodium salt/4AA |
| 5 | DHBS/4AA | 515 nm | 2.6 × 10⁴ | 3,5-Dichloro-2-hydroxybenzenesulfonate, |
| | | | | disodium salt/4AA or 3,5-Dichloro-2- |
| | | | | hydroxybenzenesulfonic acid/4AA |
| 6 | ADPS/4AA | 540 nm | 2.79 × 10⁴ | N-Ethyl-N-(3-sulfopropyl)-3-methoxyaniline, |
| | | | | sodium salt, monohydrate/4AA |
| 7 | ADOS/4AA | 542 nm | 2.72 × 10⁴ | N-Ethyl-N-(2-hydroxy-3-sulfopropyl)-3- |
| | | | | methoxylaniline, sodium salt, dehydrate/ |
| | | | | 4AA |
| 8 | DAOS/4AA | 593 nm | 1.75 × 10⁴ | N-Ethyl-N-(2-hydroxy-3-sulfopropyl)-3,5- |
| | | | | dimethoxyaniline, sodium salt/4AA |
| 9 | MADB/4AA | 630 nm | 1.65 × 10⁴ | N,N-Bis(4-sulfobutyl)-3,5-dimethylaniline, |
| | | | | disodium salt/4AA |
| 10 | TOPS/4AA | 555 nm | 3.74 × 10⁴ | N-Ethyl-N-(3-sulfopropyl)-3-methylaniline, |
| | | | | sodium salt/4AA |
| 11 | MAOS/4AA | 630 nm | 2.25 × 10⁴ | N-Ethyl-N-(2-hydroxy-3-sulfopropyl)-3,5- |
| | | | | dimethylaniline, sodium salt, monohydrate/ |
| | | | | 4AA |
| 12 | HDAOS/4AA | 583 nm | 1.75 × 10⁴ | N-(2-Hydroxy-3-sulfopropyl)-3,5- |
| | | | | dimethoxyaniline, sodium salt/4AA |
| 13 | ALPS/4AA | 561 nm | 4.13 × 10⁴ | N-Ethyl-N-(3-sulfopropyl)-aniline, sodium |
| | | | | salt/4AA |
| 14 | 4-MC/4AA | 475 nm | | 4-Methylcatechol/4AA |
| 15 | DMA/MBTH | 590 nm | | N,N-dimethylaniline (DMA)/3-Methyl-2- |
| | | | | benzothiazolinone hydrazone (MBTH) |
| 16 | TODB/MBTH | 590 nm | | N,N-Bis(4-sulfobutyl)-3-methylaniline, |
| | | | | disodium salt (TODB)/3-Methyl-2- |
| | | | | benzothiazolinone hydrazone (MBTH) |
| 17 | ALPS/MBTH | 600 nm | | N-Ethyl-N-(3-sulfopropyl)-aniline, sodium |
| | | | | salt (ALPS)/3-Methyl-2-benzothiazolinone |
| | | | | hydrazone (MBTH) |
| 18 | Amplex | 568 nm | 5.7 × 10⁴ | N-Acetyl-3,7-dihydroxyphenoxazine |
| | | | | (Amplex ® UltraRed) |
| 19 | DA67 | 666 nm | 9.0 × 10⁴ | 10-(Carboxymethylaminocarbonyl)-3,7- |
| | | | | bis(dimethylamino)-phenothiazine sodium |
| | | | | salt |
| 20 | DA64 | 727 nm | 9.0 × 10⁴ | N-(Carboxymethylaminocarbonyl)-4,4′- |
| | | | | bis(dimethylamino)-diphenylamine sodium |
| | | | | salt |
| 21 | TDBA | | | Tetradecyldimethylbenzylammonium |
| | | | | chloride |
| 22 | KNO₂ | 665 nM | | Potassium nitrite |
| 23 | DAB | 490 nm | | 3,3′-Diaminobenzidine |
| 24 | SAT-3 | 675 nm | 7.0 × 10⁴ | N,N′-Bis(2-hydroxy-3-sulfopropyl)tolidine, |
| | | | | disodium salt, |
| 25 | o-dianisidine | 450 nm | 1.15 × 10⁴ | o-Dianisidine dihydrochloride |

When the chromogenic MPO substrates were tested in the present assay, it was found that most of them either lacked adequate sensitivity or failed to minimize interferences by the plasma or serum matrix. The screening results of these chromogenic substrates are discussed in more detail in Example 3 and summarized in Table 2.

TABLE 2
Sensitivities of chromogenic MPO substrates

| | | | | | Interference |
|---|---|---|---|---|---|
| | | Wave- | | Interference | by Blood |
| | Substrate | length | Sensitivity | by Inhibitor | Components |
| 1 | Guaiacol | 470 nm | High | High false | Very high |
| | | | | negative | |
| 2 | TMB | 655 nm | High | High false | Very high |
| | | | | negative | |
| 3 | TOOS/4AA | 555 nm | Fair | Low | Low |
| 4 | TODB/4AA | 555 nm | Fair | Low | Low |
| 5 | DHBS/4AA | 515 nm | Fair | Low | Low |
| 6 | ADPS/4AA | 540 nm | Poor | N/A | N/A |
| 7 | ADOS/4AA | 542 nm | Poor | N/A | N/A |
| 8 | DAOS/4AA | 593 nm | Poor | N/A | N/A |
| 9 | MADB/4AA | 630 nm | Poor | N/A | N/A |
| 10 | TOPS/4AA | 555 nm | Fair | Low | Low |
| 11 | MAOS/4AA | 630 nm | Poor | N/A | N/A |
| 12 | HDAOS/4AA | 583 nm | Poor | N/A | N/A |
| 13 | ALPS/4AA | 561 nm | Fair | Low | Low |
| 14 | 4-MC/4AA | 475 nm | Poor | High false | N/A |
| | | | | positive | |
| 15 | DMA/MBTH | 590 nm | Poor | N/A | N/A |
| 16 | TODB/MBTH | 590 nm | Fair | High false | N/A |
| | | | | positive | |
| 17 | ALPS/MBTH | 600 nm | Fair | High false | N/A |
| | | | | positive | |
| 18 | Amplex | 568 nm | Poor | N/A | N/A |
| 19 | DA67 | 666 nm | Poor | N/A | N/A |
| 20 | DA64 | 727 nm | Poor | N/A | N/A |
| 21 | TDBA | | Poor | N/A | N/A |
| 22 | KNO₂ | 665 nM | Poor | N/A | N/A |
| 23 | DAB | 490 nm | Poor | N/A | N/A |
| 24 | SAT-3 | 675 nm | Poor | N/A | N/A |
| 25 | o-dianisidine | 450 nm | Precipitated | N/A | N/A |

As one can see from Table 2, some of the MPO substrates used in the prior art assays did not work in our system because they precipitated out of solution (o-dianisidine) or produced high interferences of MPO activity by blood components (TMB, guaiacol). Many of the substrates resulted in poor assay sensitivity or produced high numbers of false positives due to interferences by the specific MPO inhibitor in step b) of the assay, as discussed in more detail below (e.g., 4-MC/4AA, TODB/MBTH and ALPS/MBTH). The substrates that exhibited fair assay sensitivity and low interferences by blood components and the specific MPO inhibitor included TOOS/4AA, TODB/4AA, DHBS/4AA, TOPS/4AA and ALPS/4AA.

Accordingly, in some embodiments, the chromogenic MPO substrate of the present invention is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline. Preferably, the salts of N-ethyl-N-(2-hydroxy-3-sulfopropyl)-3-methylaniline, N,N-bis(4-sulfobutyl)-3-methylaniline, 3,5-dichloro-2-hydroxybenzenesulfonate, N-ethyl-N-(3-sulfopropyl)-3-methylaniline and N-ethyl-N-(3-sulfopropyl)-aniline are sodium or disodium salts.

In some embodiments, the chromogenic MPO substrate is used at a concentration that is not rate-limiting for MPO peroxidase activity. In some embodiments, the chromogenic MPO substrate is used at a final concentration that ranges from about 100 μM to about 100 mM, preferably from about 300 μM to about 30 mM, and more preferably from about 1 mM to about 10 mM.

In some embodiments, the first peroxidase activity is measured by measuring the oxidative product of the chromogenic MPO substrate. Preferably, the oxidative product of the chromogenic MPO substrate is detectable in the visible region of the electromagnetic spectrum (380-760 nm) and measured by a spectrometer or a spectrophotometer.

In some embodiments, the non-chromogenic co-substrate for MPO comprises hydrogen peroxide (H₂O₂) and/or 4-aminoantipyrine (4-AA). It is understood, however, that the nature of the non-chromogenic co-substrate is not critical to the success of the present assay, and therefore other non-chromogenic substrates can also be used.

In some embodiments, the non-chromogenic MPO co-substrate is used at a concentration that is not rate-limiting for MPO peroxidase activity. In some embodiments, H₂O₂ is used at a final concentration that ranges from about 1 μM to about 1 mM, preferably from about 10 μM to about 750 μM, and more preferably from about 100 μM to about 500 μM. In some embodiments, 4-AA is used at a final concentration that ranges from about 100 μM to about 100 mM, preferably from about 300 μM to about 30 mM, and more preferably from about 1 mM to about 10 mM.

Step b): Contacting the Blood Sample with the Chromogenic MPO Substrate, the Non-Chromogenic Co-Substrate for MPO and a Specific MPO Activity Inhibitor to Measure a Second Peroxidase Activity in the Blood Sample

In the second step of the present methods, the blood sample is contacted with the chromogenic MPO substrate, the non-chromogenic MPO co-substrate and a specific MPO inhibitor to measure non-MPO peroxidase activity in the sample. This peroxidation reaction may be summarized as follows:

Peroxidases+2AH₂+H₂O₂+MPO Inhibitor→2*AH+2H₂O(non-MPO activity) (2)

The purpose of step b) is to block MPO peroxidase activity using a specific MPO inhibitor. Preferably, the inhibitor blocks all or substantially all MPO peroxidase activity without significantly affecting the other peroxidase activities in the blood sample. As noted above, the specific MPO inhibitor can be a benzoic acid hydrazide such as 4-aminobenzoic acid hydrazide (ABAH) (Kettle et al., Biochem., 308: 559-563 (1995)), a hydroxamic acid such as benzohydroxamic acid (BHA), a salicylhydroxamic acid (SHA) (Davies & Edwards, Biochem. J., 258: 801-806 (1989)), a thioxanthine derivative such as 3-n-propyl-2-thioxanthine, 3-isobutyl-6-thioxanthine and other thioxanthine derivatives disclosed in U.S. Pat. No. 7,425,560, U.S. Pat. Appl. Nos. 2007/0032468 and 2009/0124640, Int'l Pub. Nos. WO 01/85146, WO 03/089430 and WO 05/037835, Jacobson et al., Drug. Dev. Res., 47: 45-53 (1999) and Wooldridge & Slack, J. Chem. Soc., 1863 (1962), and/or a 2,4-dihydro-[1,2,4]triazole-3-thione derivative disclosed in U.S. Pat. Appl. No. 2007/0093483, all of which are incorporated herein by reference. Preferably, the specific MPO activity inhibitor is 4-aminobenzoic acid hydrazide (ABAH).

In some embodiments, the specific MPO inhibitor is used at a concentration at which it inhibits at least about 50%, 60% or 70%, more preferably at least about 80%, 90% or 95%, and most preferably at least about 96%, 97%, 98%, 99% or 100% of the specific MPO peroxidase activity in a blood sample. In some embodiments, the final concentration of the specific MPO inhibitor ranges from about 1 μM to about 100 mM, preferably from about 10 μM to about 10 mM, and more preferably from about 100 μM to about 1 mM.

In some embodiments, the blood sample is selected from human serum or plasma; the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH). Preferably, the chromogenic MPO substrate is selected from 3,5-dichloro-2-hydroxybenzenesulfonate, a salt of 3,5-dichloro-2-hydroxybenzenesulfonate and 3,5-dichloro-2-hydroxybenzenesulfonic acid.

The enzymatic reactions in steps a) and b) are generally carried out under conditions (e.g., buffer and temperature) suitable for the completion of the enzymatic reactions. The buffer used for steps a) and b) as described herein can be the same or different. Any buffer known in the art suitable for the peroxidation reaction can be used.

In some embodiments, the enzymatic reaction may comprise additional components, such as buffers, chelating agents, stabilizers, and so forth. For example, in some embodiments, the enzymatic reaction may comprise such additional components as sodium citrate, Triton X-100, Tris-HCl, ethylene diamine tetraacetic acid (EDTA), ethylene glycol tetraacetic acid (EGTA), bovine serum albumin (BSA), sorbitol, ferrocyanide, and/or ascorbate oxidase. In some embodiments, sodium citrate is used at a final concentration ranging from about 3 mM to about 300 mM, preferably from about 10 mM to about 100 mM. In some embodiments, Triton-X is used at a final concentration ranging from about 0.003% to about 0.3%, preferably from about 0.01% to about 0.1% by weight. In some embodiments, EDTA is used at a final concentration ranging from about 3 μM to about 0.3 mM, preferably from about 10 μM to about 100 μM. In some embodiments, EGTA is used at a final concentration ranging from about 30 μM to about 3 mM, preferably from about 100 μM to about 1 mM. In some embodiments, BSA is used at a final concentration ranging from about 0.03% to about 3%, preferably from about 0.1% to about 1% by weight. In some embodiments, sorbitol is used at a final concentration ranging from about 5% to about 25%, preferably from about 10% to about 20% by weight. In some embodiments, ferrocyanide is used at a final concentration ranging from about 3 μM to about 300 μM, preferably from about 10 μM to about 100 μM. In some embodiments, ascorbate oxidase is used at a final concentration ranging from about 0.1 U/ml to about 10 U/ml, preferably from about 0.3 U/ml to about 3 U/ml.

As noted above, the pH of the peroxidation reaction has a significant effect on the assay performance because it affects both the chromogenic substrate and the enzymatic activity of MPO. For example, it was found that some of the chromogenic substrates that performed successfully in the prior art assays (e.g., o-dianisidine) precipitated from blood samples at nearly neutral pH, which is required for optimal MPO peroxidase activity. Thus, it is preferable that the present methods be carried out at approximately neutral pH values. In some embodiments, the contacting steps a) and/or b) are preferably carried out at a pH that ranges from about 5.0 to about 8.0, preferably from about 5.5 to about 7.5, and more preferably from about 6.0 to about 7.0.

The temperature for each step can be the same or different. The temperature is preferably maintained between about 25 to about 37° C.

In some embodiments, the methods of the present invention are conducted in a homogenous assay format, i.e., steps a) and b) as described herein are carried out in a single reaction mixture. Alternatively, the methods of the present invention may be conducted in a heterogeneous assay format. Preferably, the assay is automated, e.g., being conducted on a clinical analyzer; however manual operation is also possible and contemplated within the present invention.

Step c): Comparing the First and Second Peroxidase Activities to Obtain MPO Activity in the Blood Sample

In step c), MPO peroxidase activity is obtained by comparing the first peroxidase activity with the second, non-MPO peroxidase activity in the blood sample. As discussed above, the step of comparing preferably refers to quantitative comparisons such as subtracting one value from another, calculating the ratio of two values, calculating a percentage of one value with respect to another, or combining these types of calculations to produce a single number that is used as an indicator of MPO peroxidase activity. Preferably, the step of comparing the first and second peroxidase activities comprises subtracting the second peroxidase activity from the first peroxidase activity to obtain the MPO activity in the blood sample. In some embodiments, the step of comparing is performed manually by a human. Alternatively, the step of comparing may be carried out automatically by a computer or other processor, or by a combination of manual and automatic data processing.

Assays may be performed in duplicates with both positive and background controls. A standard curve can be obtained by using known amounts of myeloperoxidase with known activity. The levels of myeloperoxidase in each sample can then be determined by comparing each signal measured to the standard curve.

### C. Kits for Assaying Myeloperoxidase

The present invention also provides kits for assaying MPO peroxidase activity in a blood sample, such as a diagnostic kit. Such kits comprise a chromogenic MPO substrate that minimizes interferences of the MPO activity in a blood sample, wherein said chromogenic MPO substrate is not o-dianisidine, a non-chromogenic co-substrate for MPO, and a specific MPO activity inhibitor. Any of the chromogenic MPO substrates, non-chromogenic MPO co-substrates and specific MPO inhibitors described herein may be included in the kits.

In some embodiments, the chromogenic MPO substrate minimizes interferences of the MPO activity in a human blood sample, such as whole blood, serum or plasma from which substantially all hemoglobin has been removed, preferably serum or plasma. In some embodiments, the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline. Preferably, the salts of N-ethyl-N-(2-hydroxy-3-sulfopropyl)-3-methylaniline, N,N-bis(4-sulfobutyl)-3-methylaniline, 3,5-dichloro-2-hydroxybenzenesulfonate, N-ethyl-N-(3-sulfopropyl)-3-methylaniline and N-ethyl-N-(3-sulfopropyl)-aniline are sodium or disodium salts.

In some embodiments, the non-chromogenic co-substrate for MPO comprises hydrogen peroxide (H₂O₂) and/or 4-aminoantipyrine (4-AA). In some embodiments, the specific MPO activity inhibitor is a benzoic acid hydrazide such as 4-aminobenzoic acid hydrazide (ABAH) (Kettle et al., Biochem., 308: 559-563 (1995)), a hydroxamic acid such as benzohydroxamic acid (BHA) and salicylhydroxamic acid (SHA) (Davies & Edwards, Biochem. J., 258: 801-806 (1989)), a thioxanthine derivative such as 3-n-propyl-2-thioxanthine, 3-isobutyl-6-thioxanthine and other thioxanthine derivatives disclosed in U.S. Pat. No. 7,425,560, U.S. Pat. Appl. Nos. 2007/0032468 and 2009/0124640, Int'l Pub. Nos. WO 01/85146, WO 03/089430 and WO 05/037835, Jacobson et al., Drug. Dev. Res., 47: 45-53 (1999) and Wooldridge & Slack, J. Chem. Soc., 1863 (1962), and/or a 2,4-dihydro-[1,2,4]triazole-3-thione derivative disclosed in U.S. Pat. Appl. No. 2007/0093483, all of which are incorporated herein by reference. In preferred embodiments, the specific MPO activity inhibitor is 4-aminobenzoic acid hydrazide (ABAH).

In some embodiments, the blood sample is selected from human serum or plasma; the chromogenic MPO substrate is selected from N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH). Preferably, the chromogenic MPO substrate is selected from 3,5-dichloro-2-hydroxybenzenesulfonate, a salt of 3,5-dichloro-2-hydroxybenzenesulfonate and 3,5-dichloro-2-hydroxybenzenesulfonic acid.

The kits may further comprise positive and/or negative control standards, an apparatus or container for conducting the methods of the invention and/or transferring samples to a diagnostic laboratory for processing, as well as suitable instructions for carrying out the methods of the invention. In some embodiments, the kits may further comprise a means for measuring the oxidative product of the chromogenic MPO substrate, such as a spectrometer or a spectrophotometer capable of measuring optical signals having wavelengths in the visible region of the electromagnetic spectrum (380-760 nm).

The kits of the invention may be in any suitable packaging. For example, the packages discussed herein in relation to diagnostic systems are those customarily utilized in diagnostic systems. Such packages include containers appropriate for use in automated clinical chemistry analyzers.

D. Uses of the Methods and Kits

The present invention provides a reliable, sensitive and highly specific assay for measuring MPO peroxidase activity in a blood sample. The methods and kits of the invention thus provide a practical means for detecting conditions associated with altered levels of MPO expression and/or activity and monitoring MPO levels in a patient.

In some embodiments, the methods and kits of the invention can be used for prognosis or diagnosis of any disease associated with an inappropriate amount or reaction to myeloperoxidase present, or the effect or activity of such, in a subject. Examples of such diseases include, but are not limited to, coronary arterial disease, peripheral arterial disease, heart failure, acute myocardial infarction, acute myeloid leukemia, systemic lupus erythematosus, Hashimoto's thyroiditis, myasthenia gravis, rheumatoid arthritis, multiple sclerosis, Guillain Barre syndrome, glomerulonephritis, atherosclerosis, stroke, multiple sclerosis, Alzheimer's disease, leukemia, infection, asthma, cancer such as lung cancer, cystic fibrosis, chronic obstructive pulmonary disease, inflammatory bowel disease, neuroinflammatory diseases and microbial infections.

In further embodiments, the enzymatic methods and kits of the present invention also provide a useful research tool for the exploration of the role of myeloperoxidase in various biological processes and pathological conditions.

### EXAMPLES

### Example 1

### Assay for MPO Peroxidase Activity

To measure MPO peroxidase activity in blood samples, the following liquid stable, ready-to-use assay reagents were used:

| Reagent | Composition |
|---|---|
| Reagent 1 | 50 mM sodium citrate buffer; pH 6.0, <5 mM |
| | chromogenic substrate; and stabilizers |
| Reagent 2 | <10 mM hydrogen peroxide (H₂O₂); <20 mM 4- |
| | aminoantipyrine (4-AA); and stabilizers |
| Reagent 3 | <10 mM specific MPO inhibitor |

The detection of MPO peroxidase activity is based on the following reaction:

2H₂O₂+4-Aminoantipyrine+Chromogenic Substrate→Quinoneimine Dye+4H₂O

One MPO unit causes the hydrolysis of one micromole of hydrogen peroxide, which leads to the production of half a micromole of quinoneimine dye per minute under the conditions described below. The absorbance of quinoneimine dye can be measured at 505-515 nm.

The MPO peroxidase assay is formulated for use with non-hemolyzed lithium heparin plasma. No special handling or pretreatment is required. Plasma samples were collected such that testing could be performed as soon as possible after the specimen collection. Although the specimens may be refrigerated at 2-8° C. for 2-5 days, freezing plasma samples may lead to a decrease in MPO peroxidase activity.

In a cuvette, 140 μL of Reagent 1 and 25 μL of plasma sample were mixed and incubated at 37° C. for 1.5 minutes. Absorbance at 505 nm was read at about 2 minutes after the addition of 60 μL Reagent 2 as A1. The reaction was then incubated for approximately 3 more minutes, and the absorbance was read again as A2. The rate of the total peroxidation reaction was calculated as follows: Rate A=(A2−A1)/t, wherein t≈3 min

At about 5 minutes after the addition of Reagent 2, 46 μL of Reagent 3 was added to the reaction mix. Absorbance at 505 nm was read at about 2 minutes after the addition of Reagent 3 as A3. The reaction was then incubated for approximately 3 more minutes and the absorbance was read again as A4. The rate of the non-MPO peroxidation reaction was calculated as follows: Rate B=(A4−A3)/t, wherein t 3 min

The rate of MPO peroxidation was calculated by subtracting Rate B from Rate A:ΔRate=Rate A−Rate B. Because the specific activity of human MPO is 0.10 mU/ng, the conversion factors between the MPO mass unit and activity are as follows:
1 ng/mL MPO=0.10 mU/mL; or
1 mU/mL MPO=10.0 ng/mL

### Example 2

### Performance of the MPO Peroxidase Assay

Performance characteristic of the MPO peroxidase assay were determined using a Hitachi 917 clinical chemistry analyzer.

The performance of the MPO peroxidase assay was compared with the performance of a commercially available MPO immunoassay (CardioMPO® Immunoassay, PrognostiX, Inc.) using lithium heparin plasma samples ranging from 58 to 1095 ng/mL. For the total of 50 samples tested, the correlation coefficient between the two methods was 0.92; the slope was 0.90; and the y intercept was 17.01 ng/mL. Thus, the MPO peroxidase assay exhibited strong correlation with the predicate assay using a common clinical analyzer.

The precision of the MPO peroxidase assay was evaluated according to Clinical and Laboratory Standards Institute (formerly NCCLS) EP5-A guidelines. In the study, three levels of MPO controls containing about 105 ng/mL, 300 ng/mL, and 720 ng/mL MPO, respectively, were tested with 2 runs per day with duplicates over 20 working days. Results of these tests are summarized in Tables 3 and 4 below. The results indicate that the MPO peroxidase assay exhibited fairly low coefficients of variation within each run and between different runs.

TABLE 3
MPO assay variability within individual runs (Sᵣ)

| | Level 1: | Level 2: | Level 3: |
|---|---|---|---|
| | 105 ng/mL | 300 ng/mL | 720 ng/mL |
| | MPO | MPO | MPO |
| Number of Data Points | 80 | 80 | 80 |
| Mean (ng/mL) | 106.5 | 295.6 | 721.8 |
| SD (ng/mL) | 3.69 | 5.88 | 6.88 |
| CV (%) | 3.5% | 2.0% | 1.0% |

TABLE 4
MPO assay variability between different runs (S_{T})

| | Level 1: | Level 2: | Level 3: |
|---|---|---|---|
| | 105 ng/mL MPO | 300 ng/mL MPO | 720 ng/mL MPO |
| Number of | 80 | 80 | 80 |
| Data Points | | | |
| Mean (ng/mL) | 106.5 | 295.6 | 721.8 |
| SD (ng/mL) | 4.36 | 8.32 | 10.84 |
| CV (%) | 4.1% | 2.8% | 1.5% |

The limit of quantitation (LOQ) of the MPO peroxidase assay was determined to be 20 ng/mL, whereas the limit of blank was found to be 7.5 ng/mL. The assay maintained linearity from 20 to 1300 ng/mL (2-130.0 mU/mL) in human heparin plasma. Results below 20 ng/mL are reported as <20 ng/mL. Results that exceed 1300 ng/mL are reported as >1300 ng/mL.

Finally, effects of common substances normally present in the plasma on assay performance were tested at concentrations indicated below. Ascorbic acid, bilirubin (free), conjugated bilirubin, triglycerides, naproxen, lovastatin, ibuprofen, salicylic acid, α1-antitrypsin, and eosinophil peroxidase (EPO) produced less than 10% deviation when tested up to 0.2 mM, 40 mg/dL, 2.5 mg/dL, 1000 mg/dL, 50 mg/dL, 5.0 mg/dL, 50.0 mg/dL, 60.0 mg/dL, 13.2 mg/dL, and 5 μg/dL, respectively.

### Example 3

### Screening of Chromogenic Substrates of MPO Peroxidase

Sensitivities of various chromogenic substrates listed in Table 1 were evaluated by measuring MPO peroxidase activity in buffer solution (rate of OD change per minute, ΔOD/min) The reaction mixture contained the chromogenic substrates, as well as 0.24 mM H₂O₂ (co-substrate) and 94 ng/mL MPO in 100 mM phosphate buffer (pH 6.0-7.0). Substrate sensitivity was designated as “high” if ΔOD/min was above 0.2, “fair” if ΔOD/min was between 0.05 and 0.2, and “poor” if ΔOD/min was below 0.05. The results are summarized in Tables 2 and 5.

TABLE 5
Sensitivities of chromogenic MPO substrates

| | Substrate | Wavelength | ΔOD/min |
|---|---|---|---|
| 1 | Guaiacol | 470 nm | 0.37 |
| 2 | TMB | 655 nm | 0.32 |
| 3 | TOOS/4AA | 555 nm | 0.12 |
| 4 | TODB/4AA | 555 nm | 0.07 |
| 6 | ADPS/4AA | 540 nm | 0.004 |
| 7 | ADOS/4AA | 542 nm | 0.04 |
| 8 | DAOS/4AA | 593 nm | 0.025 |
| 9 | MADB/4AA | 630 nm | 0.04 |
| 10 | TOPS/4AA | 555 nm | 0.055 |
| 11 | MAOS/4AA | 630 nm | 0.045 |
| 12 | HDAOS/4AA | 583 nm | 0.03 |
| 18 | Amplex | 568 nm | 0.018 |
| 19 | DA67 | 666 nm | 0.023 |
| 20 | DA64 | 727 nm | 0.0012 |
| 21 | TDBA | | 0 |
| 22 | KNO₂ | 665 nM | No signal |
| 23 | DAB | 490 nm | 0.0093 |
| 24 | SAT-3 | 675 nm | 0 |
| 25 | o-dianisidine | 450 nm | Precipitated in buffer, |
| | | | pH 7.0 |

Interference by blood components was measured by spiking MPO in plasma or serum. The reaction mixture contained the chromogenic substrates and plasma or serum with spiked MPO in 100 mM phosphate buffer (pH 6-7.0). The absorption changes were monitored in the absence of the co-substrate (H₂O₂). Interference by blood components was designated as “low” if the absorption changed by ≦0.005 ΔOD/min and “high” if the absorption changed by >0.05 ΔOD/min. The results are summarized in Table 2 above.

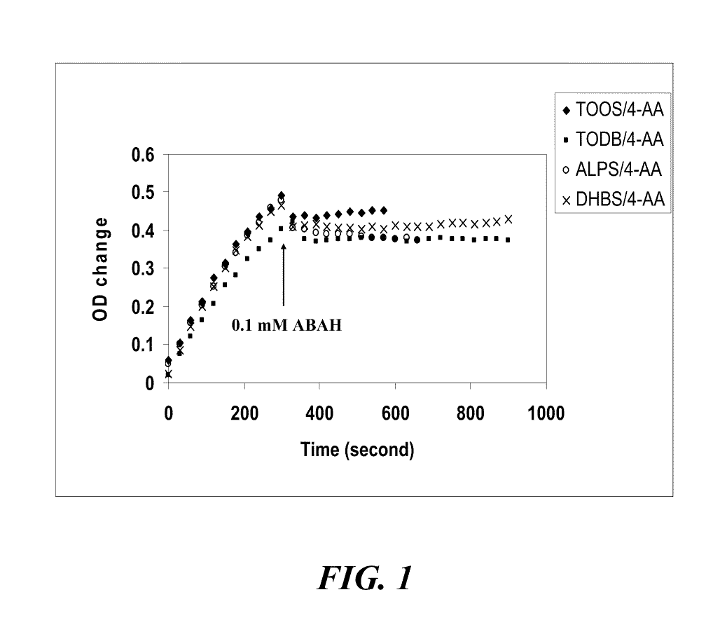

Interference by a specific MPO inhibitor was evaluated by adding 0.1 mM of 4-aminobenzoic acid hydrazide (ABAH) to the formed dye (i.e. the colored product of the substrate oxidation) in the buffer solution. Before the addition of the inhibitor, the reaction mixture contained the chromogenic substrates, as well as 0.2 mM H₂O₂ (co-substrate) and 47 ng/mL MPO in 100 mM phosphate buffer (pH 6.0-7.0). Interference by the inhibitor was designated as “low” if the absorption changed by ≦0.01 ΔOD/min and “high” if the absorption changed by >0.01 ΔOD/min. The results are summarized in Tables 2 and 6, and the corresponding kinetic traces are shown on FIG. 1. As one can see from the data, none of the four chromogenic substrates tested were associated with significant interference.

TABLE 6
Effect of specific MPO inhibitor on peroxidase activity

| | | ΔOD/min | ΔOD/min |
|---|---|---|---|
| MPO Substrate | Wavelength | (before inhibitor) | (after inhibitor) |
| TOOS/4AA | 555 nm | 0.087 | 0.004 |
| TODB/4AA | 550 nm | 0.077 | 0.001 |
| ALPS/4AA | 561 nm | 0.086 | −0.003 |
| DHBS/4AA | 515 nm | 0.089 | 0.002 |

### Example 4

### Single Channel MPO Assay

Clinical Significance. Myeloperoxidase (MPO) is a hemoprotein present in leukocytes of blood circulation. MPO is an enzyme catalyzing the hydrogen peroxide mediated peroxidation of halide ions to produce strong reactive oxidant species such as hypochlorous acid that are of potent antimicrobial activities against a broad range of invading parasites and pathogens. MPO plays therefore an important role in the innate host-defense mechanism of human and animals. However, MPO-derived reactive oxidants also promote host tissue injury through lipid and protein peroxidations that lead to cardiovascular inflammation. It is well known that elevated levels of plasma MPO is a sensitive clinical indicator of cardiovascular and other chronic inflammatory disorders¹⁻⁶. Studies also showed that elevated blood levels of MPO were associated with increased risk of stroke⁷ and angina⁸ and myocardial infarction (MI)⁹.

Assay Principle. In this exemplary single channel MPO enzymatic assay, myeloperoxidase activity is obtained in a two-step reaction by subtracting non-MPO peroxidation from total peroxidation: In the first step, total peroxidation rate A is measured through the reaction of total peroxidases in the sample with hydrogen peroxide, 4-AA and chromogenic substrate, e.g., DHBS, in the reagent; in the second step, an MPO specific inhibitor (e.g., ABAH) is added to the reaction mixture to obtain the non-MPO activity. As the result of the two-step reaction, the specific MPO activity is obtained from subtracting the non-MPO activity from the total peroxidase activity, or equals to the reaction rate A (first step) minus the reaction rate B (second step) (see illustration below).

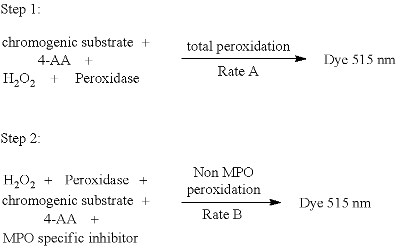

MPO activity=Total peroxidation rate A−Non MPO peroxidation rate B

Materials Required But Not Provided. Any instrument with temperature control of 37±0.5° C. that is capable of reading absorbance accurately at 515 nm may be used. Diazyme Calibrator set (Catalog No. DZ178A-CAL) and Diazyme Control set (Catalog No. DZ178A-CON) are sold separately.

Reagent Composition. See Table 7 below.

TABLE 7
Reagent Composition

| | | |
|---|---|---|
| Reagent 1 | 50-100 | mM Na-Citrate buffer pH 6.0 |
| (R1) | <5 | mM chromogenic substrate and stabilizers |
| Reagent 2 | <10 | mM H2O2 |
| (R2) | <20 | mM 4-AA and stabilizers |
| Reagent 3 | <10 | mM Inhibitor |
| (R3) | | |

Reagent Preparation. Diazyme's MPO Assay Reagents (R1, R2, and R3) are liquid stable, ready-to-use reagents.

Reagent Stability and Storage. DO NOT FREEZE. The reagents are stable when stored at 2-8° C. until the expiration date on the label. Do not mix reagents of different lots.

Specimen Collection and Preparation. The Diazyme MPO Enzymatic Assay is formulated for use with non-hemolyzed lithium heparin plasma. Collect whole blood using venipuncture techniques. Gently mix the blood with the anticoagulant by inverting sample tube several times (DO NOT SHAKE!). Place freshly collected blood samples on ice or in a refrigerator (2-8° C.) immediately, and store them at 2-8° C. until separation. Plasma should be physically separated from cells within 2 hr of collection by centrifugation at 2-8° C. Refer to the centrifugation conditions recommended by the manufacture of the specimen collection tube. Special precaution needs to be taken to ensure transfer of the plasma layer to a polypropylene (not glass) tube while avoiding carryover of any red blood cells or buffy coat white cells. The plasma samples thus prepared may be refrigerated at 2-8° C. for 3 days. DO NOT FREEZE SAMPLES.

If plasma samples are prepared by gel based vacuum tubes, the plasma samples need to be transferred to separated tubes from the top of the gels immediately after the centrifugation. Increased levels of MPO may be observed if the plasma samples are left on the top of the gels for more than 8 hours before transferring to separated tubes. New reference values need to be established if plasma samples have to be left on the top of gels for more than 8 hours before use.

Note: Plasma specimens and all materials coming in contact with them should be handled and disposed as if capable of transmitting infection. Avoid contact with skin by wearing gloves and proper laboratory attire.

Precautions. Hemolyzed samples are not suitable for use. Human source material used in calibrators and controls was tested and found negative for HIV1, HIV2, HBV, and HCV using FDA approved methods. Specimens containing human sourced materials should be handled as if potentially infectious using safe laboratory procedures, such as those outlined in Biosafety in Microbiological and Biomedical Laboratories (HHS Publication Number [CDC] 93-8395). Avoid ingestion and contact with skin or mucous membranes. See Material Safety Data Sheet. To obtain a MSDS. Do not use the reagents after the expiration date labeled on the outer box.

Assay Scheme for Chemistry Analyzers. Assay scheme for the automated chemistry analyzer Hitachi 917 is shown in the following diagram. Parameter settings for automated chemistry analyzers are available upon request (see illustration below).

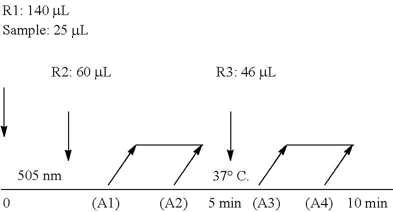

Calibration. This assay should be calibrated using Diazyme calibrators (Catalog No. DZ178A-CAL). MPO concentration in sample is determined from a linear calibration curve obtained from the MPO calibrators. Biweekly calibration is recommended. MPO calibrators are provided in freeze-dried powder form and are stable up to expiration date when stored at −20° C. Reconstitute with 0.5 mL distilled cold water carefully according to instructions on accompanying lot-specific information. Refer to the calibrator package insert for preparation. The reconstituted calibrators are stable for one day at 2-8° C.

Quality Control. Good laboratory practice recommends the use of control materials. Users should follow the appropriate federal, state and local guidelines concerning the running of external quality control(s). To ensure adequate quality control, normal and abnormal controls with known values should be run as unknown samples. Diazyme provides three levels of lyophilized MPO controls. The reconstituted MPO controls are stable for three days at 2-8° C.

Reference Range. MPO levels in heparin plasma from healthy individuals range from 19 to 160 ng/mL, with a median concentration of 135 ng/mL (937 μM or 13.5 mU/mL), based on our internal laboratory test results. A median concentration of 127 ng/mL in the plasma samples from normal subjects has been reported in the literature (Morrow D et al. European Heart Journal, 2008, 1096-1102). It is recommended that each laboratory establish its own reference range based on its patient population.

Based on the molecular weight of 144 kDa¹⁰, the following conversion factors may be used for pmol/L and ng/mL: 1 μmol/L=0.144 ng/mL or 1 ng/mL=6.94 μmol/L. The specific activity of human MPO is 0.10 mU/ng. The conversion factor between the MPO mass unit (ng/mL) and activity (miliUnit/mL) is as following: 1 ng/mL MPO=0.10 mU/mL; or 1 mU/mL MPO=10.0 ng/mL.

Limitations. The assay is designed for use with non-hemolyzed lithium heparin plasma. There is a possibility that some substances that are not listed below may interfere with the test.

Performance Characteristics. All performance characteristics were determined at Diazyme Laboratories using a Hitachi 917 chemistry analyzer.

Correlation. The performance of this assay was compared with the performance of a legally marketed MPO immunoassay using lithium heparin plasma samples ranging from 58 to 1095 ng/mL (403-7599 μmol/L). For the total of 50 samples tested, the correlation coefficient between the two methods is 0.9181; the slope is 0.9021; and y intercept is 17.009 ng/mL.

Precision. The precision of the Diazyme MPO Enzymatic Assay was evaluated according to Clinical and Laboratory Standards Institute (formerly NCCLS) EP5-A guideline. In the study, three levels of MPO controls containing about 105 ng/mL, 300 ng/mL, and 720 ng/mL MPO respectively were tested with 2 runs per day with duplicates over 20 working days (See following Tables 8 and 9).

TABLE 8
Within Run Precision (Sᵣ)

| | Level 1: | Level 2: | Level 3: |
|---|---|---|---|
| | 105 ng/mL MPO | 300 ng/mL MPO | 720 ng/mL MPO |
| Number of | 80 | 80 | 80 |
| Data Points | | | |
| Mean (ng/mL) | 106.5 | 295.6 | 721.8 |
| SD (ng/mL) | 3.69 | 5.88 | 6.88 |
| CV (%) | 3.5% | 2.0% | 1.0% |

TABLE 9
Within-Laboratory Precision (S_{T})

| | Level 1: | Level 2: | Level 3: |
|---|---|---|---|
| | 105 ng/mL MPO | 300 ng/mL MPO | 720 ng/mL MPO |
| Number of | 80 | 80 | 80 |
| Data Points | | | |
| Mean (ng/mL) | 106.5 | 295.6 | 721.8 |
| SD (ng/mL) | 4.36 | 8.32 | 10.84 |
| CV (%) | 4.1% | 2.8% | 1.5% |

Limit of Detection. The sensitivity of the Diazyme MPO enzymatic assay was determined to 13.1 ng/mL (91 pmol/L).

Limit of Quantitation. The limit of quantitation (LOQ) of the Diazyme MPO enzymatic assay was determined to be 20 ng/mL (139 μmol/L). The limit of blank was determined to be 7.5 ng/mL (52 pmol/L).

Linearity. The linearity of the assay is from 20-1300 ng/mL (139-9022 pmol/L or 2.0-130.0 mU/mL) in human heparin plasma. Results below 20 ng/mL are reported as <20 ng/mL. Results that exceed 1300 ng/mL are reported as >1300 ng/mL.

Interference. In summary, the following substances normally present in the plasma were tested at levels equal to the concentrations listed below. Ascorbic acid, bilirubin (free), conjugated bilirubin, triglycerides, naproxen, lovastatin, ibuprofen, salicylic acid, α1-antitrypsin, eosinophil peroxidase (EPO) produced less than 10% deviation when tested up to 0.2 mM, 40 mg/dL, 2.5 mg/dL, 1000 mg/dL, 50 mg/dL, 5.0 mg/dL, 50.0 mg/dL, 60.0 mg/dL, 13.2 mg/dL, and 5 μg/dL, respectively.

### REFERENCES

1. Nilsson L. et al. (1988) Activation of inflammatory system during cardiopulmonary bypass. Scand. J. Thorac. Cardovasc. Surg. 22: 51-53.
2. Heinecke J. W. et al. (1999) Mechanisms of oxidative damage by myeloperoxidase in atherosclerosis and other inflammatory disorders. J. Lab. Clin. Med. 133: 321-325.
3. Podil'chak M. D. and Terletskaia L. M. (1988) Clinical value of determining myeloperoxidase and alkaline phosphatase activity of the leukocytes in patients with suppuractive inflammatory processes. Klin. Khir. 1: 59-60.
4. Renz M., Ward M., Eastwood M. A. and Harkness R. A. (1976) Letter: Neutrophil function and myeloperoxidase activity in inflammatory bowel disease. Lancet 2(7985):584.
5. Carlsen K. H. (1997) Markers of airway inflammation in preschool wheezers. Monaldi Arch. Chest Dis. 52(5): 455-460.
6. Trush M. A., Egner P. A. and Kensler T. W. (1994) Myeloperoxidase as a biomarker of skin irritation and inflammation. Food Chem. Toxicol. 32(2): 143-147.
7. Re G. and Azzimondi G. et al (1997) Plasma lipoperoxidative markers in ischaemic stroke suggest brain embolism. European Journal of Emergency Medicine 4: 5-9.
8. Biasucci L. M., D'Onofrio G., Liuzzo G., et al.(1996) Intracellular neutrophil myeloperoxidase is reduced in unstable angina and acute myocardial infarction, but its reduction is not related to ischemia, Journal of the American College of Cardiology, 27(3): 611-616.
9. Terletskaya L. M. (1989) Granulocyte alkaline phosphatase and myeloperoxidase in patients with ischemic heart disease,” Vrach. Delo, 3:13-14.
10. Arnhold J. (2004) Properties, functions, and secretion of human myeloperoxidase. Biochemistry (Mosc.), 69(1): 4-9.

### Example 5

### Dual Channel MPO Assay

Assay Principle. In this exemplary dual channel MPO enzymatic assay, myeloperoxidase activity is obtained through two reactions run on two channels. In the first channel, total peroxidation rate A is measured through the reaction of total peroxidases in the sample with hydrogen peroxide, 4-AA and chromogenic substrate, e.g., DHBS, in the reagent; in the second channel, an MPO specific inhibitor (e.g., ABAH) is added to the reaction mixture to obtain the non MPO activity or rate B. The specific MPO activity is obtained by subtracting the non-MPO activity from the total peroxidase activity, or the reaction rate A (first reaction) minus the reaction rate B (second reaction) (see illustration below).

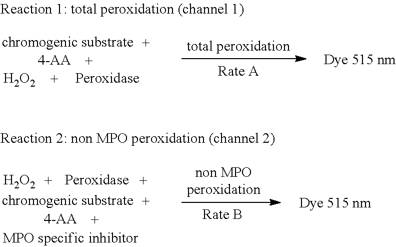

MPO activity=Total peroxidation rate A−Non MPO peroxidation rate B

Materials Required But Not Provided. Any instrument with temperature control of 37±0.5° C. that is capable of reading absorbance accurately at 515 nm may be used. Diazyme Calibrator set (Catalog No. DZ178B-CAL) and Diazyme Control set (Catalog No. DZ178B-CON) are sold separately.

Reagent Composition. See following Table 10

TABLE 10
Reagent Composition

| | | |
|---|---|---|
| Total | 50-100 | mM Na-Citrate buffer pH 6.0 |
| Peroxidation | <5 | mM chromogenic substrate and stabilizers |
| Reagent 1 | | |
| (TR1) | | |
| Non MPO | 50-100 | mM Na-Citrate buffer pH 6.0 |
| Peroxidation | <5 | mM chromogenic substrate and stabilizers |
| Reagent 1 | <10 | mM Inhibitor |
| (NR1) | | |
| Reagent 2 (R2) | <10 | mM H2O2 |
| | <20 | mM 4-AA and stabilizers |

Reagent Preparation. Diazyme's MPO Assay Reagents (TR1, NR1, and R2) are liquid stable, ready-to-use reagents. Use reagent TR1 and R2 in the first channel for total peroxidase activity (Rate A) and reagent NR1 and R2 in the second channel for non-MPO peroxidase activity (rate B).

Reagent Stability and Storage. DO NOT FREEZE. The reagents are stable when stored at 2-8° C. until the expiration date on the label. Do not mix reagents of different lots.

Specimen Collection and Preparation. The Diazyme MPO Enzymatic Assay is formulated for use with non-hemolyzed lithium heparin plasma.

Collect whole blood using venipuncture techniques. Gently mix the blood with the anticoagulant by inverting sample tube several times (DO NOT SHAKE!). Place freshly collected blood samples on ice or in a refrigerator (2-8° C.) immediately, and store them at 2-8° C. until separation. Plasma should be physically separated from cells within 2 hr of collection by centrifugation at 2-8° C. Refer to the centrifugation conditions recommended by the manufacture of the specimen collection tube. Special precaution needs to be taken to ensure transfer of the plasma layer to a polypropylene (not glass) tube while avoiding carryover of any red blood cells or buffy coat white cells. The plasma samples thus prepared may be refrigerated at 2-8° C. for 3 days. DO NOT FREEZE SAMPLES.

If plasma samples are prepared by gel based vacuum tubes, the plasma samples need to be transferred to separated tubes from the top of the gels immediately after the centrifugation. Increased levels of MPO may be observed if the plasma samples are left on the top of the gels for more than 8 hours before transferring to separated tubes. New reference values need to be established if plasma samples have to be left on the top of gels for more than 8 hours before use.

Note: Plasma specimens and all materials coming in contact with them should be handled and disposed as if capable of transmitting infection. Avoid contact with skin by wearing gloves and proper laboratory attire.

Precautions. Hemolyzed samples are not suitable for use. Human source material used in calibrators and controls was tested and found negative for HIV1, HIV2, HBV, and HCV using FDA approved methods. Specimens containing human sourced materials should be handled as if potentially infectious using safe laboratory procedures, such as those outlined in Biosafety in Microbiological and Biomedical Laboratories (HHS Publication Number [CDC] 93-8395). Avoid ingestion and contact with skin or mucous membranes. See Material Safety Data Sheet. Do not use the reagents after the expiration date labeled on the outer box.

Assay Scheme for Chemistry Analyzers. Assay scheme for the automated chemistry analyzer Hitachi 917 is shown in the following diagram. Parameter settings for automated chemistry analyzers are available upon request.

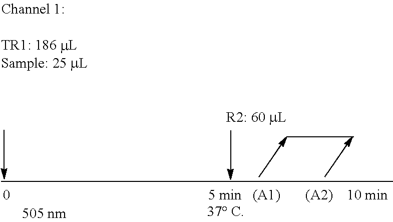

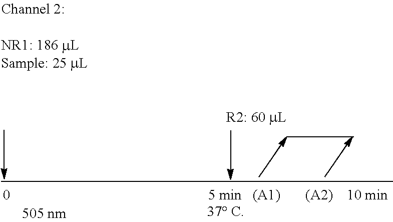

Calibration. This assay should be calibrated using Diazyme calibrators (Catalog No. DZ178B-CAL). Two levels of calibrators are included in each calibrator kit: Two vials labeled TS1 and TS2 values are for total peroxidase activity; the same two vials labeled NS1 and NS2 values are for non-MPO activity. The specific MPO activity is obtained by subtracting the non MPO activity from the total peroxidase activity. Biweekly calibration is recommended. MPO calibrators are provided in freeze-dried powder form and are stable up to expiration date when stored at −20° C. Reconstitute with 0.5 mL distilled cold water carefully according to instructions on accompanying lot-specific information. Refer to the calibrator package insert for preparation. The reconstituted calibrators are stable for one day at 2-8° C.

Quality Control. Good laboratory practice recommends the use of control materials. Users should follow the appropriate federal, state and local guidelines concerning the running of external quality control(s). To ensure adequate quality control, normal and abnormal controls with known values should be run as unknown samples. The MPO controls are provided in freeze-dried powder form and are stable up to expiration date when stored at −20° C. Three vials with three levels of MPO values are included in each control kit. Reconstitute with 0.5 mL distilled cold water carefully according to instructions on accompanying lot-specific information. Refer to the control package insert for preparation. The reconstituted MPO controls are stable for three days at 2-8° C.

The present invention is further illustrated by the following exemplary embodiments:

1. A method for measuring a myeloperoxidase (MPO) activity in a blood sample, which method comprises:

a) contacting a blood sample containing or suspected of containing MPO with a chromogenic MPO substrate that minimizes interferences of the MPO activity in said blood sample, and a non-chromogenic co-substrate for MPO to measure a first peroxidase activity in said blood sample, wherein said chromogenic MPO substrate is not o-dianisidine;

b) contacting said blood sample with said chromogenic MPO substrate, said non-chromogenic co-substrate for MPO and a specific MPO activity inhibitor to measure a second peroxidase activity in said blood sample; and

c) comparing said first and second peroxidase activities to obtain the MPO activity in said blood sample.

2. The method of embodiment 1, wherein the blood sample is a whole blood, serum or plasma sample from which substantially all hemoglobin has been removed.

3. The method of embodiment 1, wherein the blood sample is a human blood sample.

4. The method of embodiment 3, wherein the human blood sample is a human whole blood, serum or plasma sample from which substantially all hemoglobin has been removed.

5. The method of embodiment 1, wherein the chromogenic MPO substrate minimizes interferences of the MPO activity in a human blood sample.

6. The method of embodiment 5, wherein the human blood sample is a human serum or plasma sample.

7. The method of any of embodiments 1-6, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline.

8. The method of embodiment 7, wherein the salt of N-ethyl-N-(2-hydroxy-3-sulfopropyl)-3-methylaniline is a sodium salt.

9. The method of embodiment 7, wherein the salt of N,N-bis(4-sulfobutyl)-3-methylaniline is a disodium salt.

10. The method of embodiment 7, wherein the salt of 3,5-dichloro-2-hydroxybenzenesulfonate is a disodium salt.

11. The method of any of embodiments 1-10, wherein the non-chromogenic co-substrate for MPO comprises hydrogen peroxide (H₂O₂) and/or 4-aminoantipyrine (4-AA).

12. The method of any of embodiments 1-11, wherein the specific MPO activity inhibitor is selected from the group consisting of 4-aminobenzoic acid hydrazide (ABAH), benzohydroxamic acid (BHA) and salicylhydroxamic acid (SHA).

13. The method of embodiment 12, wherein the specific MPO activity inhibitor is 4-aminobenzoic acid hydrazide (ABAH).

14. The method of any of embodiments 1-13, wherein the first peroxidase activity and/or the second peroxidase activity is measured by measuring the oxidative product of the chromogenic MPO substrate.

15. The method of embodiment 14, wherein the oxidative product of the chromogenic MPO substrate is measured by spectrometry.

16. The method of any of embodiments 1-15, wherein in step c), comparing the first and second peroxidase activities comprises subtracting the second peroxidase activity from the first peroxidase activity to obtain the MPO activity in the blood sample.

17. The method of embodiment 1, wherein the blood sample is a human serum or plasma sample; the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH).

18. The method of embodiment 17, wherein the chromogenic MPO substrate is 3,5-dichloro-2-hydroxybenzenesulfonate, a salt of 3,5-dichloro-2-hydroxybenzenesulfonate or 3,5-dichloro-2-hydroxybenzenesulfonic acid.

19. The method of any of embodiments 1-18, wherein steps a) and/or b) are conducted at a pH that ranges from about 5.0 to about 8.0.

20. The method of any of embodiments 1-18, wherein the pH ranges from about 5.5 to about 7.5.

21. The method of any of embodiments 1-18, wherein the pH ranges from about 6.0 to about 7.0.

22. The method of any of embodiments 1-21, which is conducted in a homogenous or heterogeneous format.

23. The method of any of embodiments 1-22, which is automated.

24. The method of any of embodiments 1-23, wherein the method is used for prognosis and/or diagnosis of a disease.

25. The method of embodiment 24, wherein the disease is selected from the group consisting of coronary arterial disease, peripheral arterial disease, heart failure, acute myocardial infarction, atherosclerosis, stroke, multiple sclerosis, Alzheimer's disease, lung cancer, leukemia and microbial infection.

26. A kit for measuring a myeloperoxidase (MPO) activity in a blood sample, which kit comprises:

a) a chromogenic MPO substrate that minimizes interferences of the MPO activity in a blood sample, wherein said chromogenic MPO substrate is not o-dianisidine;

b) a non-chromogenic co-substrate for MPO; and

c) a specific MPO activity inhibitor.

27. The kit of embodiment 26, wherein the chromogenic MPO substrate minimizes interferences of the MPO activity in a human blood sample.

28. The kit of embodiment 26 or 27, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline.

29. The kit of embodiment 28, wherein the salt of N-ethyl-N-(2-hydroxy-3-sulfopropyl)-3-methylaniline is a sodium salt.

30. The kit of embodiment 28, wherein the salt of N,N-bis(4-sulfobutyl)-3-methylaniline is a disodium salt.

31. The kit of embodiment 28, wherein the salt of 3,5-dichloro-2-hydroxybenzenesulfonate is a disodium salt.

32. The kit of any of embodiments 26-31, wherein the non-chromogenic co-substrate for MPO comprises hydrogen peroxide (H₂O₂) and/or 4-aminoantipyrine (4-AA).

33. The kit of any of embodiments 26-32, wherein the specific MPO activity inhibitor is selected from the group consisting of 4-aminobenzoic acid hydrazide (ABAH), benzohydroxamic acid (BHA) and salicylhydroxamic acid (SHA).

34. The kit of embodiment 33, wherein the specific MPO activity inhibitor is 4-aminobenzoic acid hydrazide (ABAH).

35. The kit of embodiment 26, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH).

36. The kit of embodiment 35, wherein the chromogenic MPO substrate is 3,5-dichloro-2-hydroxybenzenesulfonate, a salt of 3,5-dichloro-2-hydroxybenzenesulfonate or 3,5-dichloro-2-hydroxybenzenesulfonic acid.

37. The kit of any of embodiments 26-36, further comprising a means for measuring the oxidative product of the chromogenic MPO substrate.

38. The kit of embodiment 37, wherein the means for measuring the oxidative product of the chromogenic MPO substrate comprise a spectrometer or a spectrophotometer.

39. The kit of any of embodiments 26-38, further comprising instructions indicating use for prognosis and/or diagnosis of a disease.

40. The kit of embodiment 39, wherein the disease is selected from the group consisting of coronary arterial disease, peripheral arterial disease, heart failure, acute myocardial infarction, atherosclerosis, stroke, multiple sclerosis, Alzheimer's disease, lung cancer, leukemia and microbial infection.

41. A method for measuring a myeloperoxidase (MPO) activity in a blood sample, which method comprises:

a) contacting a blood sample containing or suspected of containing MPO with a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline, and a non-chromogenic co-substrate for MPO to measure a first peroxidase activity in said blood sample;

b) contacting said blood sample with said chromogenic MPO substrate, said non-chromogenic co-substrate for MPO and a specific MPO activity inhibitor to measure a second peroxidase activity in said blood sample; and

c) comparing said first and second peroxidase activities to obtain the MPO activity in said blood sample.

42. A kit for measuring a myeloperoxidase (MPO) activity in a blood sample, which kit comprises:

a) a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline;

b) a non-chromogenic co-substrate for MPO; and

c) a specific MPO activity inhibitor.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A method for measuring a myeloperoxidase (MPO) activity in a blood sample, which method comprises:
a) contacting a blood sample containing or suspected of containing MPO with a chromogenic MPO substrate that minimizes interferences of the MPO activity in said blood sample, and a non-chromogenic co-substrate for MPO to measure a first peroxidase activity in said blood sample, wherein said chromogenic MPO substrate is not o-dianisidine;
b) contacting said blood sample with said chromogenic MPO substrate, said non-chromogenic co-substrate for MPO and a specific MPO activity inhibitor to measure a second peroxidase activity in said blood sample; and
c) comparing said first and second peroxidase activities to obtain said MPO activity in said blood sample.

2. The method of claim 1, wherein the human blood sample is a human whole blood, serum or plasma sample from which substantially all hemoglobin has been removed.

3. The method of claim 1, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline.

4. The method of claim 1, wherein the non-chromogenic co-substrate for MPO comprises hydrogen peroxide (H₂O₂) and/or 4-aminoantipyrine (4-AA).

5. The method of claim 1, wherein the specific MPO activity inhibitor is selected from the group consisting of 4-aminobenzoic acid hydrazide (ABAH), benzohydroxamic acid (BHA) and salicylhydroxamic acid (SHA).

6. The method of claim 1, wherein in step c), comparing the first and second peroxidase activities comprises subtracting the second peroxidase activity from the first peroxidase activity to obtain the MPO activity in the blood sample.

7. The method of claim 1, wherein the blood sample is a human serum or plasma sample; the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH).

8. The method of claim 7, wherein the chromogenic MPO substrate is 3,5-dichloro-2-hydroxybenzenesulfonate, a salt of 3,5-dichloro-2-hydroxybenzenesulfonate or 3,5-dichloro-2-hydroxybenzenesulfonic acid.

9. The method of claim 1, wherein the method is used for prognosis, diagnosis and/or monitoring treatment of a disease.

10. The method of claim 9, wherein the disease is selected from the group consisting of coronary arterial disease, peripheral arterial disease, heart failure, acute myocardial infarction, atherosclerosis, stroke, multiple sclerosis, Alzheimer's disease, lung cancer, leukemia and microbial infection.

11. A kit for measuring a myeloperoxidase (MPO) activity in a blood sample, which kit comprises:
a) a chromogenic MPO substrate that minimizes interferences of the MPO activity in a blood sample, wherein said chromogenic MPO substrate is not o-dianisidine;
b) a non-chromogenic co-substrate for MPO; and
c) a specific MPO activity inhibitor.

12. The kit of claim 11, wherein the chromogenic MPO substrate minimizes interferences of the MPO activity in a human blood sample.

13. The kit of claim 11, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline.

14. The kit of claim 11, wherein the non-chromogenic co-substrate for MPO comprises hydrogen peroxide (H₂O₂) and/or 4-aminoantipyrine (4-AA).

15. The kit of claim 11, wherein the specific MPO activity inhibitor is selected from the group consisting of 4-aminobenzoic acid hydrazide (ABAH), benzohydroxamic acid (BHA) and salicylhydroxamic acid (SHA).

16. The kit of claim 11, wherein the chromogenic MPO substrate is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline; the non-chromogenic co-substrate for MPO comprises hydrogen peroxide (H₂O₂) and 4-aminoantipyrine (4-AA); and the specific MPO activity inhibitor is 4-aminobenzoic acid hydrazide (ABAH).

17. The kit of claim 11, wherein the chromogenic MPO substrate is 3,5-dichloro-2-hydroxybenzenesulfonate, a salt of 3,5-dichloro-2-hydroxybenzenesulfonate or 3,5-dichloro-2-hydroxybenzenesulfonic acid.

18. The kit of claim 11, further comprising a spectrometer or a spectrophotometer for measuring an oxidative product of the chromogenic MPO substrate.

19. The kit of claim 11, further comprising instructions indicating use for prognosis, diagnosis and/or monitoring treatment of a disease.

20. The kit of claim 19, wherein the disease is selected from the group consisting of coronary arterial disease, peripheral arterial disease, heart failure, acute myocardial infarction, atherosclerosis, stroke, multiple sclerosis, Alzheimer's disease, lung cancer, leukemia and microbial infection.

21. A method for measuring a myeloperoxidase (MPO) activity in a blood sample, which method comprises:
a) contacting a blood sample containing or suspected of containing MPO with a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline, and a non-chromogenic co-substrate for MPO to measure a first peroxidase activity in said blood sample;
b) contacting said blood sample with said chromogenic MPO substrate, said non-chromogenic co-substrate for MPO and a specific MPO activity inhibitor to measure a second peroxidase activity in said blood sample; and
c) comparing said first and second peroxidase activities to obtain said MPO activity in said blood sample.

22. A kit for measuring a myeloperoxidase (MPO) activity in a blood sample, which kit comprises:
a) a chromogenic MPO substrate that is selected from the group consisting of N-ethyl-N-(2-hydroxy-3-sulfopropyl)-3-methylaniline, a salt of N-ethyl-N-(2-hydroxy-3-sulfopropyl)-3-methylaniline, N,N-bis(4-sulfobutyl)-3-methylaniline, a salt of N,N-bis(4-sulfobutyl)-3-methylaniline, 3,5-dichloro-2-hydroxybenzenesulfonate, a salt of 3,5-dichloro-2-hydroxybenzenesulfonate, 3,5-dichloro-2-hydroxybenzenesulfonic acid, N-ethyl-N-(3-sulfopropyl)-3-methylaniline, a salt of N-ethyl-N-(3-sulfopropyl)-3-methylaniline, N-ethyl-N-(3-sulfopropyl)aniline and a salt of N-ethyl-N-(3-sulfopropyl)-aniline;
b) a non-chromogenic co-substrate for MPO; and
c) a specific MPO activity inhibitor.

23. The method of claim 1, wherein the first peroxidase activity and the second peroxidase activity are measured in a single channel sequentially, the first peroxidase activity being measured in the presence of the chromogenic MPO substrate that minimizes interferences of the MPO activity in the blood sample and the non-chromogenic co-substrate for MPO to measure a first total peroxidase activity in the blood sample, the second peroxidase activity being measured by adding the specific MPO activity inhibitor after the first peroxidase activity is measured to measure a second non-MPO peroxidase activity in the blood sample, and the MPO activity in the blood sample is obtained by subtracting the second non-MPO peroxidase activity from the first total peroxidase activity.

24. The method of claim 23, wherein the first peroxidase activity is measured after addition of reagent 1 comprising the chromogenic MPO substrate and reagent 2 comprising the non-chromogenic co-substrate for MPO to the blood sample, and the second peroxidase activity is measured after addition of reagent 3 comprising the specific MPO activity inhibitor to the blood sample after the first peroxidase activity is measured.

25. The kit of claim 11, which comprises the following reagents:
a) reagent 1 comprising the chromogenic MPO substrate;
b) reagent 2 comprising the non-chromogenic co-substrate for MPO; and
c) reagent 3 comprising the specific MPO activity inhibitor.

26. The method of claim 21, wherein the first peroxidase activity and the second peroxidase activity are measured in a single channel sequentially, the first peroxidase activity being measured in the presence of the chromogenic MPO substrate that minimizes interferences of the MPO activity in the blood sample and the non-chromogenic co-substrate for MPO to measure a first total peroxidase activity in the blood sample, the second peroxidase activity being measured by adding the specific MPO activity inhibitor after the first peroxidase activity is measured to measure a second non-MPO peroxidase activity in the blood sample, and the MPO activity in the blood sample is obtained by subtracting the second non-MPO peroxidase activity from the first total peroxidase activity.

27. The method of claim 26, wherein the first peroxidase activity is measured after addition of reagent 1 comprising the chromogenic MPO substrate and reagent 2 comprising the non-chromogenic co-substrate for MPO to the blood sample, and the second peroxidase activity is measured after addition of reagent 3 comprising the specific MPO activity inhibitor to the blood sample after the first peroxidase activity is measured.

28. The kit of claim 22, which comprises the following reagents:
a) reagent 1 comprising the chromogenic MPO substrate;
b) reagent 2 comprising the non-chromogenic co-substrate for MPO; and
c) reagent 3 comprising the specific MPO activity inhibitor.

29. The method of claim 1, wherein the first peroxidase activity and the second peroxidase activity are measured in two channels separately, the first peroxidase activity being measured in the presence of the chromogenic MPO substrate that minimizes interferences of the MPO activity in the blood sample and the non-chromogenic co-substrate for MPO in a first channel to measure a first total peroxidase activity in the blood sample, the second peroxidase activity being measured in the presence of the chromogenic MPO substrate that minimizes interferences of the MPO activity in the blood sample, the non-chromogenic co-substrate for MPO and the specific MPO activity inhibitor in a second channel to measure a second non-MPO peroxidase activity in the blood sample, and the MPO activity in the blood sample is obtained by subtracting the second non-MPO peroxidase activity from the first total peroxidase activity.

30. The method of claim 29, wherein the first peroxidase activity is measured after addition of reagent 1 comprising the chromogenic MPO substrate and reagent 3 comprising the non-chromogenic co-substrate for MPO to the blood sample, and the second peroxidase activity is measured after addition of reagent 2 comprising the chromogenic MPO substrate and the specific MPO activity inhibitor and reagent 3 comprising the non-chromogenic co-substrate for MPO to the blood sample.

31. The kit of claim 11, which comprises the following reagents:
a) reagent 1 comprising the chromogenic MPO substrate;
b) reagent 2 comprising the chromogenic MPO substrate and the specific MPO activity inhibitor; and
c) reagent 3 comprising the non-chromogenic co-substrate for MPO.

32. The method of claim 21, wherein the first peroxidase activity and the second peroxidase activity are measured in two channels separately, the first peroxidase activity being measured in the presence of the chromogenic MPO substrate that minimizes interferences of the MPO activity in the blood sample and the non-chromogenic co-substrate for MPO in a first channel to measure a first total peroxidase activity in the blood sample, the second peroxidase activity being measured in the presence of the chromogenic MPO substrate that minimizes interferences of the MPO activity in the blood sample, the non-chromogenic co-substrate for MPO and the specific MPO activity inhibitor in a second channel to measure a second non-MPO peroxidase activity in the blood sample, and the MPO activity in the blood sample is obtained by subtracting the second non-MPO peroxidase activity from the first total peroxidase activity.

33. The method of claim 32, wherein the first peroxidase activity is measured after addition of reagent 1 comprising the chromogenic MPO substrate and reagent 3 comprising the non-chromogenic co-substrate for MPO to the blood sample, and the second peroxidase activity is measured after addition of reagent 2 comprising the chromogenic MPO substrate and the specific MPO activity inhibitor and reagent 3 comprising the non-chromogenic co-substrate for MPO to the blood sample.

34. The kit of claim 22, which comprises the following reagents:
a) reagent 1 comprising the chromogenic MPO substrate;
b) reagent 2 comprising the chromogenic MPO substrate and the specific MPO activity inhibitor; and
c) reagent 3 comprising the non-chromogenic co-substrate for MPO.
